(19) United States
(12) Patent Application Publication
(10) Pub. No.: US 2014/0221464 A1
(43) Pub. Date: Aug. 7, 2014

(54) Compositions and Methods for Treating Skeletal Myopathy

(71) Applicant: Eric N. Olson, Dallas, TX (US)

(72) Inventor: Eric N. Olson, Dallas, TX (US)

(73) Assignee: BOARD OF REGENTS, THE UNIVERSITY OF TEXAS SYSTEM, Austin, TX (US)

(21) Appl. No.: 14/130,236

(22) PCT Filed: Jul. 2, 2012
(86) PCT No.: PCT/US12/45274

§ 371 Date: Mar. 24, 2014

Related U.S. Application Data
(60) Provisional application No. 61/504,048, filed on Jul. 1, 2011.

(51) Int. Cl. C07H 21/02

(52) U.S. Cl. CPC C07H 21/02
USPC 514/44 R

(57) ABSTRACT

The present invention provides a method of preventing or treating a myopathy, such as a skeletal myopathy, comprising administering a modulator of a miRNA. In one embodiment, the skeletal myopathy is centronuclear myopathy. The modulator can be an agonist that promotes the expression, function or activity of a miR-133 family member. The miR-133 family member can be miR-133a or miR-133b.

### CROSS-REFERENCE TO RELATED APPLICATIONS

This application claims priority to and the benefit of U.S. Provisional Application Ser. No. 61/504,048, filed Jul. 1, 2011, which is herein incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: MIRG_{—}029_{—}01WO_SeqList_ST25.txt, date recorded: Jul. 2, 2012, file size 5.3 kilobytes).

### FIELD OF TILE PRESENT INVENTION

The present invention relates generally to the prevention or treatment of abnormal skeletal muscle activity or function by modulating the expression or activity of a microRNA (miRNA). In particular, the activity or expression of a miR-133 family member is modulated.

### BACKGROUND OF THE PRESENT INVENTION

Skeletal myopathies are diseases of the skeletal muscle, and can be inherited or acquired. Human centronuclear myopathies (CNMs) are a group of congenital myopathies characterized by muscle weakness and abnormal centralization of nuclei in muscle myofibers (1, 2). CNMs can be classified into 3 main forms: the recessive X-linked myotubular myopathy (XLMTM), with a severe neonatal phenotype, caused by mutations in the myotubularin gene (MTM1); the classical autosomal-dominant form, with mild, moderate, or severe phenotypes, caused by mutations in the dynamin 2 gene (DNM2); and an autosomal-recessive form presenting severe and moderate phenotypes, caused by mutations in the amphiphysin 2 gene (BIN1) (1, 2). Despite their heterogeneous clinical phenotypes, all 3 forms of CNMs present the following common pathological characteristics: (a) type I myofiber predominance and small fiber sizes; (b) abnormal NADH-tetrazolium reductase (NADH-TR) staining patterns, indicative of mitochondrial abnormalities; and (c) absence of necrosis, myofiber death, or regeneration (2).

XLMTM, the most severe and most common form of CNM, has been extensively studied in mice and zebrafish (3-6). Mice with homozygous mutations of the Mtm1 gene develop a progressive CNM that recapitulates the pathological characteristics of XLMTM in humans (5). Mtnm1-deficient mice also display disorganized triads and defective excitation-contraction coupling, which may be responsible for the impaired muscle function in XLMTM (3).

The autosomal-dominant form of CNM is associated with a wide clinical spectrum of slowly progressive CNMs, from those beginning in childhood or adolescence to more severe sporadic forms with neonatal onset (7-9). Multiple missense mutations in the DNM2 locus have been identified in recent years, hence, the autosomal-dominant CNM is also called DNM2-associated CNM. Dynamin 2 is a ubiquitously expressed large GTPase involved in many cellular functions, including endocytosis and membrane trafficking (10, 11). However, the precise mechanism whereby multiple missense mutations in the DNM2 gene cause CNM remains unknown. Furthermore, there is no mouse model for DNM2-related CNM, and a knockin mouse model expressing the most frequent CNM-related DNM2 mutation, R465W Dnm2, failed to reproduce the autosomal-dominant form of human CNM (9). Homozygous mice carrying the R465W Dnm2 mutation die within 24 hours after birth, whereas heterozygous mice develop a myopathy followed by atrophy and impaired muscle function without centralized nuclei (9).

MicroRNAs modulate cellular phenotypes by inhibiting expression of mRNA targets. microRNAs (miRNAs) are highly conserved small noncoding RNAs that regulate a range of biological processes by inhibiting the expression of target mRNAs with complementary sequences in their 3′ untranslated regions (3′ UTRs) (12). Watson-Crick base pairing of nucleotides 2-8 of a miRNA with the mRNA target results in mRNA degradation and/or translational repression. Recent studies have revealed roles for miRNAs in the regulation of skeletal muscle differentiation, and changes in miRNA expression are associated with various skeletal muscle disorders (13-15). However, the involvement of miRNAs in skeletal myopathies has not been demonstrated. Identification and characterization of miRNAs involved in myopathies is important for the development of novel therapeutic approaches for the treatment of myopathies, such as skeletal myopathies, including CNM.

### SUMMARY OF THE PRESENT INVENTION

The present invention is based, in part, on the discovery that miRNA have an essential role in the maintenance of skeletal muscle structure, function, bioenergetics, and myofiber identity. Accordingly, disclosed herein are methods and compositions for treating or preventing a skeletal myopathy. In one particular embodiment, the skeletal myopathy is a centronuclear myopathy (CNM). In one embodiment, a method for treating or preventing a CNM in a subject in need thereof comprises administering to the subject an agonist of a miR-133 family member. Also provided herein is a method of maintaining skeletal muscle structure or function, inhibiting fast-to-slow myofiber conversion, or treating or preventing mitochondrial dysfunction in a subject in need thereof comprising administering to the subject an agonist of a miR-133 family member.

The miR-133 family member can be miR-133a or miR-133b. For example, the agonist is a polynucleotide comprising a miR-133a or miR-133b sequence. The polynucleotide can comprise a pri-miR-133a, pre-miR-133a, or mature miR-133a sequence. In another embodiment, the polynucleotide comprises a pri-miR-133b, pre-miR-133b, or mature miR-133b sequence. For example, the polynucleotide can comprise a sequence of 5′-UUUGGUCCCCUUCAACCAGCUG-3′ (SEQ ID NO: 2) or 5′-UUUGGUCCCCUUCAACCAGCUA-3′ (SEQ ID NO: 4).

The agonist can be a polynucleotide formulated in a lipid delivery vehicle. In some embodiments, the polynucleotide is encoded by an expression vector. The polynucleotide can be under the control of a skeletal muscle promoter, such as the muscle creatine kinase promoter. In one embodiment, the polynucleotide is double-stranded. In another embodiment, the polynucleotide is conjugated to cholesterol. The polynucleotide can be about 70 to about 100 nucleotides in length. In some embodiments, the polynucleotide is about 18 to about 25 nucleotides in length.

In some embodiments, the agonist is administered to the subject by a subcutaneous, intravenous, intramuscular, or intraperitoneal route of administration. The subject can be a human. In some embodiments, the subject has a mutation in the myotubularin (MTM1) gene, dynamin 2 (DNM2) gene, and/or amphiphysin 2 (BIN1) gene.

The present invention also provides a method for identifying a modulator of a miR-133 family member in skeletal muscle comprising: (a) contacting a skeletal muscle cell with a candidate compound; (b) assessing the activity or expression of the miR-133 family member, and (c) comparing the activity or expression in step (b) with the activity or expression in the absence of the candidate compound, wherein a difference between the measured activities or expression indicates that the candidate compound is a modulator of the miR-133 family member. The miR-133 family member can be miR-133a or miR-133b, and the cell contacted with the candidate compound in vitro or in vivo. The candidate compound can be a peptide, polypeptide, polynucleotide, or small molecule. Assessing the activity of the miR-133 family can comprise determining T-tubule organization, mitochondrial function, DNM2 expression, or type I myofiber composition.

### BRIEF DESCRIPTION OF THE DRAWINGS

The following drawings form part of the present specification and are included to further demonstrate certain aspects of the present invention. The present invention may be better understood by reference to one or more of these drawings in combination with the detailed description of specific embodiments presented herein.

FIG. 1. Expression of miR-133 in skeletal muscle. (A) Northern blot analysis of miR-133a in adult WT mouse tissues. The blot was stripped and reprobed with 32P-labeled U6 probe as a loading control. Sol, soleus. (B) Expression of miR-133 in skeletal muscle, detected by real-time RT-PCR and expressed relative to U6.

FIG. 2. dKO mice have normal muscle appearance at four weeks of age. (A) H&E staining of soleus, EDL, G/P and TA muscles from WT and dKO mice at 4 weeks of age. Scale bar=40 μm. (B) TA muscle from WT and dKO mice at 4 weeks of age was immunostained with antibody against laminin. DAPI stain was used to detect nuclei and showed no centralized nuclei. Size bar: 30 μm. (C) Cross-sectional areas of TA muscle fibers of WT and dKO mice at 4 weeks of age was determined using ImageJ program. n=3 WT and dKO. More than 300 TA fibers from each mouse were examined.

FIG. 3. Characterization of dKO mice. (A) Percentage of centronuclear fibers in various muscle groups of WT and dKO mice at 6-8 weeks of age. n=3 for WT and n=6 for dKO. Error bars represent SEM. (B) Measurements of body mass (BW) and muscle mass relative to tibia length (TL) ratios from WT and dKO mice at 12 weeks of age. ** represents p<0.01; *** represents p<0.001. (C) Cross-sectional areas of TA muscle fibers were determined from WT and dKO mice at 3 months of age. n=5 for WT and n=7 for dKO.

FIG. 4. Centronuclear myofibers in dKO skeletal muscle. (A) H&E staining of soleus, EDL, G/P, and TA muscles of WT and dKO mice at 12 weeks of age. Scale bars: 40 μm. (B) Immunostaining of TA muscle against laminin. Nuclei are stained with DAPI. dKO TA muscle showed central nuclei. Scale bars: 40 μm. (C) Percentage of centronuclear myofibers in 4 WT mice and 10 dKO mice at 12 weeks of age. For each mouse, more than 500 myofibers were counted for TA and G/P muscles and more than 300 myofibers were counted for soleus and EDL muscles. (D) NADH-TR staining of dKO TA muscle revealed abnormal distribution, radiating intermyofibrillar network (arrows), and ring-like fibers (asterisks). Scale bars: 20 μm. (E) EBD uptake of TA muscles of WT, dKO, and mdx mice. Immunostaining with laminin (green) is shown; EBD is detected as a red signal under fluorescence microscopy. Scale bars: 100 μm. (F) Expression of myogenic genes and of embryonic MHC (Myh3) and perinatal MHC (Myh8) in WT and dKO TA muscle, determined by real-time RT-PCR. n=3 (WT and dKO).

FIG. 5. Analysis of dKO muscles by NADH-TR, H&E and immunohistochemistry. (A) NADH-TR staining of soleus, EDL, G/P, and TA muscles of WT and dKO mice at 12 weeks of age. Scale bar=40 μm. (B) NADH-TR staining of soleus, EDL, G/P, and TA muscles of WT and dKO mice at 4 weeks of age. Scale bar=40 μm. (C) H&E staining of TA muscle of WT and dKO mice at 12 months of age. Scale bar=40 μm. (D) Immunostaining of TA muscle from WT and dKO mice at 4 weeks using antibody against DHPRα to detect T-tubule distributions. There is no apparent difference in the T-tubule staining pattern between WT and dKO muscle at this age. Size bar: 30 μm.

FIG. 6. Disorganization of triads in TA muscle fibers in dKO mice. (A) Expression of mRNA transcripts of encoding components of T-tubules and SR was determined by real-time RT-PCR in TA muscles of 12-week-old mice. n=3 (WT and dKO). (B) Immunostaining of T-tubules and SR in transverse sections of TA muscle from WT and dKO mice at 12 weeks of age. T-tubules were detected by anti-DHPRα, and terminal cisternae of the SR were detected by anti-RyR1. Nuclei were detected by DAPI, and the myofiber perimeter was stained by anti-laminin. Images of multiple levels of the sections were taken and reconstructed to create the 3D effect. Scale bars: 30 μm. (C-J) Electron micrographs of WT and dKO muscle. dKO TA muscle showed accumulation of electron-dense structures (D-F) that were absent in WT TA muscle (C). dKO muscle (H and J) displayed T-tubules (arrows) in abnormal orientations compared with WT muscle (G and I). Scale bars: 2 μm (C and D); 0.5 μm (E-H); 0.2 μm (I and J).

FIG. 7. Western blot analysis of WT and dKO TA muscle on proteins related to SR and T-tubules. Western blot analysis was performed on protein lysates from 3 month-old WT dKO TA muscle. Antibodies were used to detect expression of RyR1, DPHRα, Calsequestrin (Casq), SERCA2, Phospholamban (pln), phosphorylated Phospholamban at Serine 16 (Ser16-pln), Sarcolipin (sin), CamKII, and phosphorylated CamKII. α-actin was detected as a loading control.

FIG. 8. Mitochondrial dysfunction in dKO muscle. (A) Mitochondria were isolated from red and white gastrocnemius muscle, and oxygen consumption rate (OCR) was measured for RCR, ADP-stimulated state 3 respiration (ADP), and FCCP-stimulated respiration (FCCP). n=2 (WT and dKO). *P<0.05 vs. WT. (B) Fatty acid oxidation was measured in isolated mitochondria from red and white gastrocnemius muscle. Citrate synthase enzyme activity was measured in isolated mitochondria from red and white quadriceps muscle. n=6 (WT and dKO). *P<0.05 vs. WT.

FIG. 9. miR-133a regulates Dnm2 expression in skeletal muscle. (A) Position of miR-133a target site in Dnm2 3′ UTR and sequence alignment of miR-133a (5′-UUGGUCCCCUUCAACCAGCUA-3′ (SEQ ID NO: 29)) and the Dnm2 3′ UTR from mouse (5′-UGCCCUCCAUGCUGGGACCAGGCUCCCCG-3′ (SEQ ID NO: 30)), human (5′-CGCCCCUAUGCUGGGACCAGGCUCCCAG-3′ (SEQ ID NO: 31)), and rat (5′-UGCCCCCCAUGCUGGGACCAGGCUCCCCG-3′ (SEQ ID NO: 32)) are shown. Conserved miR-133a binding sites in Dnm2 3′ UTR (5′-GGGACCA-3′ (SEQ ID NO: 33)) is shown. Mutations in Dnn2 3′ UTR were introduced to disrupt base-pairing with miR-133a seed sequence (5′-UGGUCCC-3′ (SEQ ID NO: 34)). (B) Luciferase reporter constructs containing WT and mutant Dnm2 3′ UTR sequences were cotransfected into COS-1 cells with a plasmid expressing miR-133a. 48 hours after transfection, luciferase activity was measured and normalized to β-galactosidase activity. (C) Real-time RT-PCR showing expression of Dnm2 mRNA in WT and dKO TA muscle. n=3 (WT and dKO). (D) Western blot showing expression of dynamin 2 protein in TA muscle of WT and dKO mice. n=2 (WT and dKO). The blot was stripped and reprobed with an antibody against α-actin as a loading control. Quantification of dynamin 2 protein, determined by densitometry and normalized to α-actin, is also shown.

FIG. 10. Overexpression of Dnm2 in skeletal muscle causes CNM. (A) Western blot analysis of TA muscle from WT and MCK-DNM2 transgenic mouse lines Tg1 and Tg2 using anti-dynamin 2 and anti-myc to show overexpression of transgene. Anti-tubulin was used as a loading control. Protein quantification, determined by densitometry, is also shown. (B) Transverse sections of TA muscles of WT, Tg1, and Tg2 mice at 6 weeks of age were stained with wheat germ agglutinin (WGA) and DAPI to show central nuclei (arrows) in transgenic mice. Scale bars: 100 μm. (C) Percentage of centronuclear myofibers in TA muscle of transgenic mice at 7 weeks of age. (D) Histological analysis of TA and soleus muscles of WT and Tg2 mice at 11 weeks of age. TA muscle sections were stained with H&E, anti-laminin, and DAPI to show central nuclei and with NADH-TR to reveal abnormal distribution and radiating intermyofibrillar network (arrows). Scale bars: 40 μm. (E) 10-week-old WT and Tg2 mice (n=3 per group), as well as 3 month-old WT and dKO mice (n=5 per group), were subjected to forced downhill running on a treadmill. Muscle performance was measured as time to exhaustion. Total running distance is also shown. *P<0.05; ***P<0.001.

FIG. 11. Analysis of MCK-Dnm2 transgenic mice. (A) Measurements of body mass (BW) and muscle mass of WT and MCK-Dnm2 Tg mice at 11 weeks of age. ** represents p<0.01; *** represents p<0.001. n=3 for WT and Tg2 mice. (B) Immunostaining of TA muscle from WT and Tg2 mice at 11 weeks of age using antibody against DHPRα to detect T-tubule distributions. Size bar: 30 μm. (C) Top panel: western blot analysis showing expression of dynamin 2 protein in Tg2 soleus muscle and heart at 11 weeks of age. Bottom panel: histological analysis of soleus muscle of WT and Tg2 mice at 11 weeks of age. Soleus muscle sections were stained with H&E and Metachromatic ATPase to show Type I myofibers (dark blue).

FIG. 12. Intracellular accumulation of dysferlin in dKO and MCK-DNM2 transgenic mouse myofibers. (A) Immunostaining of TA muscle from WT and dKO mice to detect dynamin 2 and dysferlin. Intracellular accumulation of dysferlin was observed in dKO myofibers. Overlay images indicate localization of dynamin 2 and dysferlin in the intracellular aggregates in dKO muscle. Scale bars: 30 μm. (B) Immunostaining of TA muscle from WT and Tg2 mice to detect dysferlin. Intracellular accumulation of dysferlin was observed in Tg2 myofibers. Scale bars: 30 μm.

FIG. 13. Control of skeletal muscle fiber type by miR-133a. (A) Metachromatic ATPase staining and anti-MHC-1 immunostaining of soleus muscle from WT and dKO mice at 12 weeks of age showed an increase in type I myofibers in dKO soleus muscle. H&E staining of the soleus muscles is also shown. Scale bars: 100 μm. (B) Percentage of type I myofibers in soleus muscles, determined by metachromatic ATPase staining. n=6 (WT and dKO). (C) Expression of transcripts of MHC isoforms in soleus muscle, determined by real-time RT-PCR. n=3 (WT and dKO). Expression of MHC isoforms from protein extracts of soleus, EDL, and TA muscles from WT and dKO mice was also determined by glycerol gel electrophoresis followed by silver staining.

FIG. 14. Fiber type analysis of WT and dKO muscles. (A) Immunohistochemistry of soleus and EDL muscles from WT and dKO mice at postnatal day 1 using antibody against MHC-I. Scale bar=100 μm. (B) Metachromatic ATPase staining of soleus muscle from WT and dKO mice at 4 and 2 weeks. Scale bar=100 μm.

### DETAILED DESCRIPTION OF THE PRESENT INVENTION

The present invention is based, in part, on the discovery that miRNA have an essential role in the maintenance of skeletal muscle structure, function, bioenergetics, and myofiber identity. Accordingly, disclosed herein are methods and compositions for treating or preventing abnormal skeletal muscle function or activity, such as a skeletal myopathy. Through the creation of mice with genetic deletions of miR-133a-1 and miR-133a-2, the inventors developed a mouse model for CNM, in which the mice developed adult-onset CNM. The mice developed CNM in type II (fast-twitch) myofibers accompanied by impaired mitochondrial function, fast-to-slow myofiber conversion, and disarray of muscle triads (sites of excitation-contraction coupling). These abnormalities mimic human CNMs and could be ascribed, at least in part, to dysregulation of the miR-133a target mRNA that encodes dynamin 2, a GTPase implicated in human centronuclear myopathy. Thus, the inventors have established that miR133 family members, in particular, miR-133a-1 and miR-133a-2, are essential for multiple facets of skeletal muscle function and homeostasis. Accordingly, the present invention provides novel therapeutic approaches for treating and preventing abnormal skeletal muscle function or activity by modulating the activity or expression of a miR-133 family member.

The miR-133 family contains 3 highly homologous miRNAs: miR-133a-1, miR-133a-2, and miR-133b. The miR-1-1/miR-133a-2 and miR-1-2/miR-133a-1 miRNA clusters are expressed in cardiac and skeletal muscle, whereas the miR-206/miR-133b cluster is only expressed in skeletal muscle (16). MiR-206 is required for efficient regeneration of neuromuscular synapses after acute nerve injury, and loss of miR-206 accelerates disease progression of amyotrophic lateral sclerosis in mice (17). MiR-1 and miR-133a play important roles in heart development and function (18, 19) and have also been shown to regulate myoblast proliferation and differentiation in vitro (20), however, the potential functions of these miRNAs in skeletal muscle development or function in vivo were not studied.

MiR-133a-2 is co-transcribed with miR-1-1 from human chromosome 20, while miR-133a-1 is co-transcribed with miR-1-2 from human chromosome 18. MiR-133b is generated with miR-206 from a bicistronic transcript from an intergenic region of human chromosome 6. MiR-133a-1 and miR-133a-2 are identical to each other and differ from miR-133b by two nucleotides (18). MiR-133a-1 and miR-133a-2 are expressed in cardiac and skeletal muscle, whereas miR-133b is skeletal muscle specific (18). The stem-loop and mature sequences for miR-133a, and miR-133b are shown below:

Human miR-133a stem-loop (SEQ ID NO: 1):
ACAAUGCUUUGCUAGAGCUGGUAAAAUGGAACCAAAUCGCCUCUUCAAUG
GAUUUGGUCCCCUUCAACCAGCUGUAGCUAUGCAUUGA
Human mature miR-133a (SEQ ID NO: 2):
UUUGGUCCCCUUCAACCAGCUG
Human miR-133b stem-loop (SEQ ID NO: 3):
CCUCAGAAGAAAGAUGCCCCCUGCUCUGGCUGGUCAAACGGAACCAAGUC
CGUCUUCCUGAGAGGUUUGGUCCCCUUCAACCAGCUACAGCAGGGCUGGC
AAUGCCCAGUCCUUGGAGA
Human mature miR-133b (SEQ ID NO: 4):
UUUGGUCCCCUUCAACCAGCUA

The present invention provides a method of treating or preventing a centronuclear myopathy in a subject in need thereof comprising administering to the subject an agonist of a miR-133 family member. Also provided is a method of maintaining skeletal muscle structure or function, inhibiting fast-to-slow myofiber conversion, and preventing or treating mitochondrial dysfunction in a skeletal muscle cell in a subject in need thereof comprising administering to the subject an agonist of a miR-133 family member.

An “agonist” can be any compound or molecule that increases the activity or expression of the particular miRNA. For example, in certain embodiments, an agonist of a miR-133 family member is a polynucleotide comprising a mature miR-133a or miR-133b sequence. In some embodiments, the polynucleotide comprises the sequence of SEQ ID NO: 2, and/or SEQ ID NO: 4. In another embodiment, the agonist of a miR-133 family member can be a polynucleotide comprising the pri-miRNA or pre-miRNA sequence for a miR-133 family member, such as for miR-133a or miR-133b. In such an embodiment, the polynucleotide can comprise a sequence of SEQ ID NO: 1 and/or SEQ ID NO: 3. The polynucleotide comprising the mature sequence, the pre-miRNA sequence, or the pri-miRNA sequence for miR-133a or miR-133b can be single stranded or double stranded. In some embodiments, the polynucleotide is at least about 75%, 80%, 85%, 90%, 95%, 96%, 97%, 98%, or 99% complementary to the mature sequence, the pre-miRNA sequence, or the pri-miRNA sequence for miR-133a or miR-133b. In one embodiment, the polynucleotide comprises a sequence that is 100% complementary to the mature sequence, the pre-miRNA sequence, or the pri-miRNA sequence for miR-133a or miR-133b.

The polynucleotides can contain one or more chemical modifications, such as locked nucleic acids, peptide nucleic acids, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages and combinations comprising the same. In one embodiment, the polynucleotide comprising a miR-133a or miR-133b sequence is conjugated to a steroid, such as a cholesterol, a vitamin, a fatty acid, a carbohydrate or glycoside, a peptide, or another small molecule ligand. In another embodiment, the agonist of miR-133a or miR-133b can be an agent distinct from miR-133a or miR-133b that acts to increase, supplement, or replace the function of miR-miR-133a or miR-133b.

In another embodiment, the agonist of miR-133a or miR-133b can be expressed in vivo from a vector. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the present invention.

In one embodiment, an expression vector for expressing an agonist of miR-133a or miR-133b comprises a promoter “operably linked” to a polynucleotide encoding miR-133a or miR-133b, such as the mature sequence, the pre-miRNA sequence, or the pri-miRNA sequence for miR-133a or miR-133b. The phrase “operably linked” or “under transcriptional control” as used herein means that the promoter is in the correct location and orientation in relation to a polynucleotide to control the initiation of transcription by RNA polymerase and expression of the polynucleotide. The polynucleotide encoding miR-133a or miR-133b may encode the primary miRNA sequence (pri-miRNA), the precursor-miRNA sequence (pre-miRNA), or the mature miRNA sequence for miR-133a or miR-133b. In some embodiments, the polynucleotide encodes a polynucleotide that is at least about 75%, 80%, 85%, 90%, 95%, 96%, 97%, 98%, or 99% complementary to the mature sequence, the pre-miRNA sequence, or the pri-miRNA sequence for miR-133a or miR-133b. In one embodiment, the polynucleotide encodes a polynucleotide that is 100% complementary to the mature sequence, the pre-miRNA sequence, or the pri-miRNA sequence for miR-133a or miR-133b.

In another embodiment, the expression vector comprises a polynucleotide operably linked to a promoter, wherein said polynucleotide comprises the sequence of SEQ ID NO: 1. In some embodiments, the polynucleotide comprises a sequence that is at least about 75%, 80%, 85%, 90%, 95%, 96%, 97%, 98%, 99%, or 100% complementary to SEQ ID NO. 1. In another embodiment, the expression vector comprises a polynucleotide operably linked to a promoter, wherein said polynucleotide comprises the sequence of SEQ ID NO: 2. In some embodiments, the polynucleotide comprises a sequence that is at least about 75%, 80%, 85%, 90%, 95%, 96%, 97%, 98%, 99%, or 100% complementary to SEQ ID NO. 2. In another embodiment, the expression vector comprises a polynucleotide operably linked to a promoter, wherein said polynucleotide comprises the sequence of SEQ ID NO: 3. In some embodiments, the polynucleotide comprises a sequence that is at least about 75%, 80%, 85%, 90%, 95%, 96%, 97%, 98%, 99%, or 100% complementary to SEQ ID NO. 3. In another embodiment, the expression vector comprises a polynucleotide operably linked to a promoter, wherein said polynucleotide comprises the sequence of SEQ ID NO: 4. In some embodiments, the polynucleotide comprises a sequence that is at least about 75%, 80%, 85%, 90%, 95%, 96%, 97%, 98%, 99%, or 100% complementary to SEQ ID NO. 4.

The polynucleotide comprising the sequence of SEQ ID NO: 1, SEQ ID NO: 2, SEQ ID NO: 3, or SEQ ID NO: 4 may be about 18 to about 2000 nucleotides in length, about 70 to about 200 nucleotides in length, about 20 to about 50 nucleotides in length, or about 18 to about 25 nucleotides in length. In some embodiments, the polynucleotide comprising a sequence that is at least about 75%, 80%, 85%, 90%, 95%, 96%, 97%, 98%, 99%, or 100% complementary to SEQ ID NO. 1, 2, 3, or 4 is about 18 to about 2000 nucleotides in length, about 70 to about 200 nucleotides in length, about 20 to about 50 nucleotides in length, or about 18 to about 25 nucleotides in length.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III.

In some embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the polynucleotide sequence of interest. The use of other viral or mammalian cellular or bacterial phage promoters which are well-known in the art to achieve expression of a polynucleotide sequence of interest is contemplated as well, provided that the levels of expression are sufficient for a given purpose. In certain embodiments, a tissue-specific promoter, such as a skeletal muscle-specific promoter, can be used to obtain tissue-specific expression of the polynucleotide sequence of interest.

By employing a promoter with well-known properties, the level and pattern of expression of the protein of interest following transfection or transformation can be optimized. Further, selection of a promoter that is regulated in response to specific physiologic signals can permit inducible expression of the polynucleotide. Several regulatory elements that may be employed, in the context of the present invention, to regulate the expression of the polynucleotide of interest (e.g. agonists of miR-133a or miR-133b).

Viral promoters, cellular promoters/enhancers and inducible promoters/enhancers that could be used in combination with the polynucleotide of interest in an expression construct.

Additionally, any promoter/enhancer combination (as per the Eukaryotic Promoter Data Base EPDB) could also be used to drive expression of the polynucleotide. Eukaryotic cells can support cytoplasmic transcription from certain bacterial promoters if the appropriate bacterial polymerase is provided, either as part of the delivery complex or as an additional genetic expression construct.

The following list is not intended to be exhaustive of all the possible elements involved in the promotion of expression of a polynucleotide of interest, merely, to be exemplary thereof. Examples of promoters or enhancers that can be used include, but are not limited to, the following (or derived from the following): Immunoglobulin Heavy Chain, Immunoglobulin Light Chain, T-Cell Receptor, HLA DQ a and/or DQ β,β-Interferon, Interleukin-2, Interleukin-2 Receptor, MHC Class II 5, MHC Class II HLA-DRa, β-Actin, Muscle Creatine Kinase (MCK), Prealbumin (Transthyretin), Elastase 1, Metallothionein (MTII), Collagenase, Albumin, α-Fetoprotein, t-Globin, β-Globin, c-fos, c-HA-ras, Insulin, Neural Cell Adhesion Molecule (NCAM), α₁-Antitrypain, H2B (TH2B) Histone, Mouse and/or Type I Collagen, Glucose-Regulated Proteins (GRP94 and GRP78), Rat Growth Hormone, Human Serum Amyloid A (SAA), Troponin I (TN I), Platelet-Derived Growth Factor (PDGF), Duchenne Muscular Dystrophy, SV40, Polyoma, Retroviruses, Papilloma Virus, Hepatitis B Virus, Human Immunodeficiency Virus, Cytomegalovirus (CMV), and Gibbon Ape Leukemia Virus.

Examples of inducible elements/inducers that can be used include, but are not limited to, the following (or derived from the following): MT II/Phorbol Ester (TFA), Heavy metals; MMTV (mouse mammary tumor virus). Glucocorticoids; β-Interferon/poly(rI)x, poly(rc); Adenovirus 5 E2/E1A: Collagenase/Phorbol Ester (TPA); Stromelysin/Phorbol Ester (TPA); SV40/Phorbol Ester (TPA); Murine MX Gene/Interferon, Newcastle Disease Virus; GRP78 Gene/A23187; α-2-Macroglobulin/IL-6; Vimentin/Serum; MHC Class I Gene H-2κb/Interferon; HSP70/E1A, SV40 Large T Antigen; Proliferin/Phorbol Ester-TPA; Tumor Necrosis Factor/PMA; and Thyroid Stimulating Hormone α Gene/Thyroid Hormone.

Of particular interest are muscle specific promoters, which include, but are not limited to, the myosin light chain-2 promoter (Franz et al. (1994) Cardioscience, Vol. 5(4):235-43; Kelly et al. (1995) J. Cell Biol., Vol. 129(2):383-396), alpha actin promoter (Moss et al. (1996) Biol. Chem., Vol. 271(49):31688-31694), troponin 1 promoter (Bhavsar et al. (1996) Genomics, Vol. 35(1):11-23); Na+/Ca+ exchanger promoter (Barnes et al. (1997) J. Biol. Chem., Vol. 272(17):11510-11517), dystrophin promoter (Kimura et al. (1997) Dev. Growth Differ., Vol. 39(3):257-265), alpha7 integrin promoter (Ziober and Kramer (1996) J. Bio. Chem., Vol. 271(37):22915-22), brain natriuretic peptide promoter (LaPointe et al. (1996) Hypertension, Vol. 27(3 Pt 2):715-22), alpha B-crystallin/small heat shock protein promoter (Gopal-Srivastava (1995) J. Mol. Cell. Biol., Vol. 15(12):7081-7090), alpha myosin heavy chain promoter (Yamauchi-Takihara et al. (1989) Proc. Natl. Acad. Sci. USA, Vol. 86(10):3504-3508), the ANF promoter (LaPointe et al. (1988) J. Biol. Chem., Vol. 263(19):9075-9078), and the muscle creatine kinase (MCK) promoter (Jaynes et al., Mol. Cell Biol. 6: 2855-2864 (1986); Horlick and Benfield, Mol. Cell. Biol., 9:2396, 1989; Johnson et al., Mol. Cell. Biol., 9, 3393 (1989)).

A polyadenylation signal may be included to effect proper polyadenylation of the polynucleotide where desired. Any such sequence may be employed such as human growth hormone and SV40 polyadenylation signals. Also contemplated as an element of the expression cassette is a terminator. These elements can serve to enhance message levels and to minimize read through from the cassette into other sequences.

There are a number of ways in which expression vectors comprising a polynucleotide of the present invention may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells.

One of the methods for in vivo delivery involves the use of an adenovirus expression vector. “Adenovirus expression vector” is meant to include those constructs containing adenovirus sequences sufficient to (a) support packaging of the construct and (b) to express a polynucleotide that has been cloned therein. The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

In one embodiment, the vector is replication defective and will not have an adenovirus E1 region. Thus, it may be convenient to introduce the polynucleotide encoding an agonist disclosed herein at the position from which the E1-coding sequences have been removed. However, the position of insertion of the construct within the adenovirus sequences is not critical to the invention. The polynucleotide encoding the agonist of interest may also be inserted in lieu of the deleted E3 region in E3 replacement vectors, or in the E4 region where a helper cell line or helper virus complements the E4 defect. Adenovirus vectors can be administered into different tissues, such as by trachea instillation, muscle injection, peripheral intravenous injections and stereotactic inoculation into the brain.

Retroviral vectors are also suitable for expressing agonists of a miR-133 family member, such as miR-133a or miR-133b, in cells. The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a polynucleotide of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types.

Other viral vectors may be employed as expression constructs in the present invention. Vectors derived from viruses such as vaccinia virus, adeno-associated virus (AAV) and herpesviruses may be employed. They offer several attractive features for various mammalian cells.

In order to effect expression of the polynucleotide of interest (ie. agonist of a miR-133 family member), the expression construct should be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells as known in the art also are contemplated by the present invention. These include calcium phosphate precipitation, DEAE-dextran, electroporation, direct microinjection. DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell, the nucleic acid encoding the polynucleotide of interest may be positioned and expressed at different sites. In certain embodiments, the nucleic acid encoding the polynucleotide of interest may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. For example, polyomavirus DNA in the form of calcium phosphate precipitates has been delivered into the liver and spleen of adult and newborn mice demonstrating active viral replication and acute infection, and direct intraperitoneal injection of calcium phosphate-precipitated plasmids has been shown to result in expression of the transfected genes. It is envisioned that DNA encoding a polynucleotide of interest (ie. an agonist of a miR-133 family member) may also be transferred in a similar manner in vivo and expressed.

In still another embodiment of the present invention, transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads. Selected organs including the liver, skin, and muscle tissue of rats and mice have been bombarded in vivo. This may require surgical exposure of the tissue or cells, to eliminate any intervening tissue between the gun and the target organ, i.e., ex vivo treatment. Again, DNA encoding a particular polynucleotide of interest (ie. an agonist of a miR-133 family member) may be delivered via this method and still be incorporated by the present invention.

In a further embodiment of the present invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

In certain embodiments of the invention, the liposome may be complexed with a hemagglutinating virus (HVJ). This has been shown to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it may be desirable to include within the liposome an appropriate bacterial polymerase.

Other expression constructs which can be employed to deliver a nucleic acid encoding a particular agonist of a miR-133 family member into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific. Receptor-mediated gene targeting vehicles generally consist of two components: a cell receptor-specific ligand and a DNA-binding agent. Several ligands have been used for receptor-mediated gene transfer. Extensively characterized ligands are asialoorosomucoid (ASOR) and transferrin are contemplated by the present invention. A synthetic neoglycoprotein, which recognizes the same receptor as ASOR, has been used as a gene delivery vehicle and epidermal growth factor (EGF) has also been used to deliver genes to squamous carcinoma cell, which are also contemplated for use herein.

In other embodiments, the delivery vehicle may comprise a ligand and a liposome. For example, lactosyl-ceramide, a galactose-terminal asialganglioside, has been incorporated into liposomes and an increase in the uptake of the insulin gene by hepatocytes was observed. Thus, it is feasible that a nucleic acid encoding a particular gene also may be specifically delivered into a cell type by any number of receptor-ligand systems with or without liposomes.

In a particular example, the oligonucleotide may be administered in combination with a cationic lipid. Examples of cationic lipids include, but are not limited to, lipofectin, DOTMA, DOPE, and DOTAP. The publication of WO0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication Nos. 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are each incorporated by reference in its entirety.

In certain embodiments, delivery may more easily be performed under ex vivo conditions. Ex vivo refers to the isolation of cells from an animal, the delivery of a nucleic acid into the cells in vitro, and then the return of the modified cells back into an animal. This may involve the surgical removal of tissue/organs from an animal or the primary culture of cells and tissues.

In certain embodiments, the cells containing a nucleic acid construct of the present invention is to be identified. A cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding the polynucleotide of interest (ie. an agonist of a miR-133 family member). Further examples of selectable markers are well known to one of skill in the art.

In one embodiment, the present invention provides a method of treating or preventing a skeletal myopathy in a subject. A “skeletal myopathy” refers to a condition in which there is a disease to the skeletal muscle that is not caused by a nerve disorder. Myopathies can be caused by inherited genetic defects (e.g., muscular dystrophies), or by endocrine, inflammatory (e.g., polymyositis), and metabolic disorders. Symptoms can include, but are not limited to, weakening and atrophy of skeletal muscles, such as proximal muscles or distal muscles. Some myopathies, such as the muscular dystrophies, develop at an early age, and others develop later in life.

In some embodiments, the present invention provides a method of treating or preventing centronuclear myopathies (CNMs) comprising administering an agonist of a miR-133 family member. CNMs are a group of congenital myopathies characterized by muscle weakness and abnormal centralization of nuclei in muscle myofibers (1, 2). CNMs can be classified into 3 main forms: the recessive X-linked myotubular myopathy (XLMTM), with a severe neonatal phenotype, caused by mutations in the myotubularin gene (MTM1); the classical autosomal-dominant form, with mild, moderate, or severe phenotypes, caused by mutations in the dynamin 2 gene (DNM2); and an autosomal-recessive form presenting severe and moderate phenotypes, caused by mutations in the amphiphysin 2 gene (BIN1) (1, 2). Thus, in one embodiment, the present invention provides a method of treating or preventing XLMTM, the classical autosomal-dominant form of CNM, or the autosomal-recessive form of CNM in a subject. The method can comprise administering an agonist of a miR-133 family member, such as an agonist of miR-133a or miR-133b. In another embodiment, a method of treating or preventing CNM comprises administering a miR-133 family member, such as an agonist of miR-133a or miR-133b, to a subject with a mutation in the MTM1, DNM2, or BIN1 gene.

The characteristics of CNM typically include the following common pathological characteristics: (a) type I myofiber predominance and small fiber sizes; (b) abnormal NADH-tetrazolium reductase (NADH-TR) staining patterns, indicative of mitochondrial abnormalities; and (c) absence of necrosis, myofiber death, or regeneration (2). Thus, also provided herein is a method of maintaining skeletal muscle structure or function, inhibiting fast-to-slow myofiber conversion, or preventing or treating a mitochondrial dysfunction in a subject comprising administering an agonist of a miR-133 family member. In some embodiments, the subject is a mammal, such as a human, mouse, horse, or dog.

In another embodiment of the present invention, it is envisioned to use an agonist of a miR-133 family member in combination with other therapeutic modalities. Thus, in addition to the miRNA agonists of the present invention described herein, one may also provide to the subject “standard” pharmaceutical therapies. Such standard therapies will depend upon the particular skeletal myopathy to be treated, but can include drug therapy, physical therapy, bracing, surgery, massage and acupuncture.

Combinations may be achieved by contacting skeletal muscle cells with a single composition or pharmacological formulation that includes both agents, or by contacting the cell with two distinct compositions or formulations, at the same time, wherein one composition includes an agonist of a miR-133 family member and the other includes the second agent. Alternatively, the therapy using an miRNA agonist may precede or follow administration of the other agent(s) by intervals ranging from minutes to weeks. In embodiments where the other agent and miRNA agonists are applied separately to the cell, one would generally ensure that a significant period of time did not expire between the time of each delivery, such that the agent and miRNA agonists would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a miRNA agonist, or the other agent will be desired. In this regard, various combinations may be employed. By way of illustration, where the miRNA agonist is “A” and the other agent/therapy is “B,” the following permutations based on 3 and 4 total administrations are exemplary: A/B/A, B/A/B, B/B/A, A/A/B, B/A/A, A/B/B, B/B/B/A. B/B/A/B, A/A/B/B, A/B/A/B, A/B/B/A, B/B/A/A, B/A/B/A, B/A/A/B, B/B/B/A, A/A/A/B, B/A/A/A, A/B/A/A, A/A/B/A, A/B/B/B, B/A/B/B, and B/B/A/B. Other combinations are likewise contemplated.

The present invention also contemplates methods for scavenging or clearing agonists of a miR-133 family member following treatment. In one embodiment, the method comprises overexpression of binding site regions for a miR-133 family member in skeletal muscle cells using a muscle specific promoter. The binding site regions preferably contain a sequence of the seed region, the 5′ portion of a miRNA spanning bases 2-8, for a miR-133 family member. In some embodiments, the binding site may contain a sequence from the 3′ UTR of one or more targets of a miR-133 family member. For instance, in one embodiment, a binding site for miR-133a family member contains the 3′ UTR of DNM2. In another embodiment, an inhibitor of a miR-133 family member may be administered after an agonist of a miR-133 family member to attenuate or stop the function of the microRNA. Such inhibitors can include antagomirs, antisense, or inhibitory RNA molecules (e.g. siRNA or shRNA).

The present invention also encompasses pharmaceutical compositions comprising an agonist of a miR-133 family member and a pharmaceutically acceptable carrier, such as a miR-133a agonist and a pharmaceutically acceptable carrier or a miR-133b agonist and a pharmaceutically acceptable carrier. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Colloidal dispersion systems, such as macromolecule complexes, nanocapsules, microspheres, beads, and lipid-based systems including oil-in-water emulsions, micelles, mixed micelles, and liposomes, can be used as delivery vehicles for the agonists of microRNA function described herein. Commercially available fat emulsions that are suitable for delivering the nucleic acids of the invention to tissues, such as skeletal muscle tissue, include Intralipid™, Liposyn™, Liposyn™ II, Liposyn™ III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO 03/093449, which are herein incorporated by reference in their entireties.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the delivery vehicle, dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the nucleic acids of the compositions.

The active compositions of the present invention may include classic pharmaceutical preparations. Administration of these compositions according to the present invention may be via any common route so long as the target tissue is available via that route. This includes oral, nasal, or buccal. Alternatively, administration may be by intradermal, transdermal, subcutaneous, intramuscular, intraperitoneal or intravenous injection, or by direct injection into skeletal muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Pharmacological therapeutic agents and methods of administration, dosages, etc., are well known to those of skill in the art (see for example, the “Physicians Desk Reference,” Klaassen's “The Pharmacological Basis of Therapeutics,” “Remington's Pharmaceutical Sciences,” and “The Merck Index, Eleventh Edition,” incorporated herein by reference in relevant parts), and may be combined with the invention in light of the disclosures herein. Suitable dosages include about 20 mg/kg to about 200 mg/kg, about 40 mg/kg to about 160 mg/kg, or about 80 mg/kg to about 100 mg/kg. Some variation in dosage will necessarily occur depending on the condition of the subject being treated. The person responsible for administration will, in any event, determine the appropriate dose for the individual subject, and such individual determinations are within the skill of those of ordinary skill in the art. Moreover, for human administration, preparations should meet sterility, pyrogenicity, general safety and purity standards as required by FDA Office of Biologics standards.

Any of the compositions described herein may be comprised in a kit. In a non-limiting example, a miR-133a and/or miR-133b agonist is included in a kit. The kit may further include water and hybridization buffer to facilitate hybridization of the two strands of the miRNAs. The kit may also include one or more transfection reagent(s) to facilitate delivery of the polynucleotide agonists to cells.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

Such kits may also include components that preserve or maintain the miRNAs/polynucleotides or that protect against their degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented. A kit may also include utensils or devices for administering the miRNA agonist by various administration routes, such as parenteral or intramuscular administration.

The present invention also includes a method for diagnosing a skeletal myopathy in a subject. In one embodiment, the method comprises (a) obtaining a skeletal muscle tissue sample from the subject; (b) assessing activity or expression of a miR-133 family member in the sample; and (c) comparing the activity or expression in step (b) with the activity or expression of a miR-133 family member in a normal tissue sample, wherein an increase in the activity or expression of the miR-133 family member as compared to the activity or expression of the miR-133 family member in a normal tissue sample is diagnostic of a skeletal myopathy. The miR-133 family member can be miR-133a or miR-133b. In some embodiments, the activity or expression of both miR-133a and miR-133b are assessed. The skeletal myopathy can be CNM.

In one embodiment, assessing activity of a miR-133 family member comprises assessing the activity of one or more genes regulated by the miR-133 family member, such as one or more genes regulated by miR-133a and/or miR-133b. For instance, in some embodiments, the one or more genes regulated by miR-133a is DNM2. In another embodiment, the method further comprises administering to the subject a therapy for the skeletal myopathy and reassessing the expression or activity of miR-133a and/or miR-133b. The expression or activity of miR-133a and/or miR-133b can be obtained following treatment and compared to expression of these miRNAs in a normal tissue sample or a tissue sample obtained from the subject previously (e.g. prior to treatment).

The present invention further comprises methods for identifying modulators of skeletal muscle function. For instance, in one embodiment, the present invention provides a method for identifying a modulator of a miR-133 family member in skeletal muscle. Identified agonists of the function of the miR-133 family member are useful in the treatment or prevention of skeletal myopathies, such as CNM. Modulators (e.g. agonists) of miR-133a and/or miR-133b can be included in pharmaceutical compositions for the treatment or prevention of CNM, maintaining skeletal muscle structure or function, inhibiting fast-to-slow myofiber conversion, or preventing or treating mitochondrial dysfunction according to the methods of the present invention.

Assays for identifying a modulator may comprise random screening of large libraries of candidate substances; alternatively, the assays may be used to focus on particular classes of compounds selected with an eye towards structural attributes that are believed to make them more likely to inhibit the promote the activity or expression of a miR-133 family member.

To identify a modulator of a miR-133 family member, one can generally determine the function or activity of the miR-133 family member in the presence and absence of the candidate compound. In one embodiment, the method comprises: (a) contacting a skeletal muscle cell with a candidate compound; (b) assessing the activity or expression of a miR-133 family member, and (c) comparing the activity or expression in step (b) with the activity or expression in the absence of the candidate compound, wherein a difference between the measured activities or expression indicates that the candidate compound is a modulator of the miR-133 family member, and hence skeletal muscle function or maintenance. Assays also may be conducted in isolated cells, organs, or in living organisms.

Assessing the activity or expression of a miR-133 family member can comprise assessing the expression level of the miR-133 family member, such as the expression level of miR-133a and/or miR-133b. Those in the art will be familiar with a variety of methods for assessing RNA expression levels including, for example, northern blotting or RT-PCR. Assessing the activity or expression of the miR-133 family member can comprise assessing the activity of the miR-133 family member, such as the activity of miR-133a and/or miR-133b. In other embodiments, assessing the activity of the miR-133 family member comprises assessing expression or activity of a gene regulated by the miR-133 family member, such as regulated by miR-133a and/or miR-133b, such as DNM2. Those in the art will be familiar with a variety of methods for assessing the activity or expression of genes regulated by a miR-133 family member. Such methods include, for example, northern blotting, RT-PCR, ELISA, or western blotting.

In some embodiments, assessing the activity comprises the activity or expression of the miR-133 family member can comprise assessing T-tubule organization, mitochondrial function, DNM2 protein or gene expression, or type I myofiber composition. Those in the art will be familiar with a variety of methods, such as, but not limited to, those described in the following examples. For example, T-tubule organization can be assessed by electron microscopy, immunohistochemistry and/or examining the expression of genes encoding components of T-tubules and SR that are important for excitation-contraction coupling, including the α1, β1, and γ1 subunits of the dihydropyridine receptor (DHPR) (encoded by Cacna1s, Cacnb1, and Cacng1, respectively), ryanodine receptor 1 (Ryr1), type 1 and 2 SERCA pumps (Atp2a1 and Atp2a2), Sarcolipin, and calsequestrin 1 and 2 (Casq1 and Casq2). Mitochondrial function can be assessed by mitochondrial respiration and/or fatty acid oxidation. Assessments of mitochondrial function can include, but is not limited to: (a) respiratory control ratio (RCR), the coupling between oxidative phosphorylation and ATP synthesis; (b) ADP-stimulated state 3 respiration, the respiratory rate during which the mitochondria are producing ATP; and (c) carbonylcyanide-p-trifluoromethoxyphenylhydrazone-stimulated (FCCP-stimulated) respiration. Fiber composition can be analyzed by metachromatic ATPase staining and/or immunohistochemistry.

Fiber composition can also be assessed by quantitative real-time RT-PCR analysis of the expression of transcripts encoding individual MHC isoforms, such as type I MHC (MHC-I) and type II MHCs (MHC-IIa, MHC-IIx/d, and MHC-IIb).

It will, of course, be understood that all the screening methods of the present invention are useful in themselves notwithstanding the fact that effective candidates may not be found. The invention provides methods for screening for such candidates, not solely methods of finding them.

As used herein the term “candidate compound” refers to any molecule that may potentially modulate skeletal muscle maintenance and function by a miR-133 family member. One can acquire, from various commercial sources, molecular libraries that are believed to meet the basic criteria for useful drugs in an effort to “brute force” the identification of useful compounds. Screening of such libraries, including combinatorially-generated libraries, is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds. Non-limiting examples of candidate compounds that may be screened according to the methods of the present invention are proteins, peptides, polypeptides, polynucleotides, oligonucleotides or small molecules. Modulators of a miR-133 family member may also be agonists or inhibitors of upstream regulators of the miR-133 family member.

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads. For example, one may assess the hybridization of an oligonucleotide to a target miRNA. A technique for high throughput screening of compounds is described in WO 84/03564. Large numbers of small compounds may be synthesized on a solid substrate, such as plastic pins or some other surface. Such molecules can be rapidly screened for their ability to hybridize to miR-133a and/or miR-133b.

The present invention also contemplates the screening of compounds for their ability to modulate expression and function of a miR-133 family member in cells. Various cell lines, including those derived from skeletal muscle cells (e.g. C2C12 cells), can be utilized for such screening assays, including cells specifically engineered for this purpose.

In vivo assays involve the use of various animal models, such as a miR-133a^{-/-} mouse as described in the examples. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal derived from any of these species, including those modified to provide a model of skeletal myopathies.

Treatment of animals with test compounds will involve the administration of the compound, in an appropriate form, to the animal. Administration will be by any route that could be utilized for clinical purposes. Determining the effectiveness of a compound in vivo may involve a variety of different criteria, including but not limited to alteration of synapse architecture or signaling. Also, measuring toxicity and dose response can be performed in animals in a more meaningful fashion than in in vitro or in cyto assays.

The present invention includes a method of regulating expression of DNM2 in a cell comprising contacting the cell with a modulator of a miR-133 family member. In one embodiment, the expression of DNM2 is decreased in the cell following administration of a miR-133 (ie. miR-133a) agonist. In another embodiment, the expression of DNM2 is increased in the cell following administration of a miR-133 (ie. miR-133a) inhibitor. In certain embodiments, the cell is a skeletal muscle cell.

The following examples are included to further illustrate various aspects of the invention. It should be appreciated by those of skill in the art that the techniques disclosed in the examples which follow represent techniques and/or compositions discovered by the inventor to function well in the practice of the present invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Expression of miR-133 in Skeletal Muscle

MiR-133a-1 and miR-133a-2 are important for cardiac development and function (18). Mice lacking either miR-133a-1 or miR-133a-2 are normal, whereas approximately 50% of double knockout (dKO) mice lacking both miRNAs die as embryos or neonates from ventricular-septal defects (18). To explore the functions of miR-133a in skeletal muscle, the surviving miR-133a dKO mice were studied.

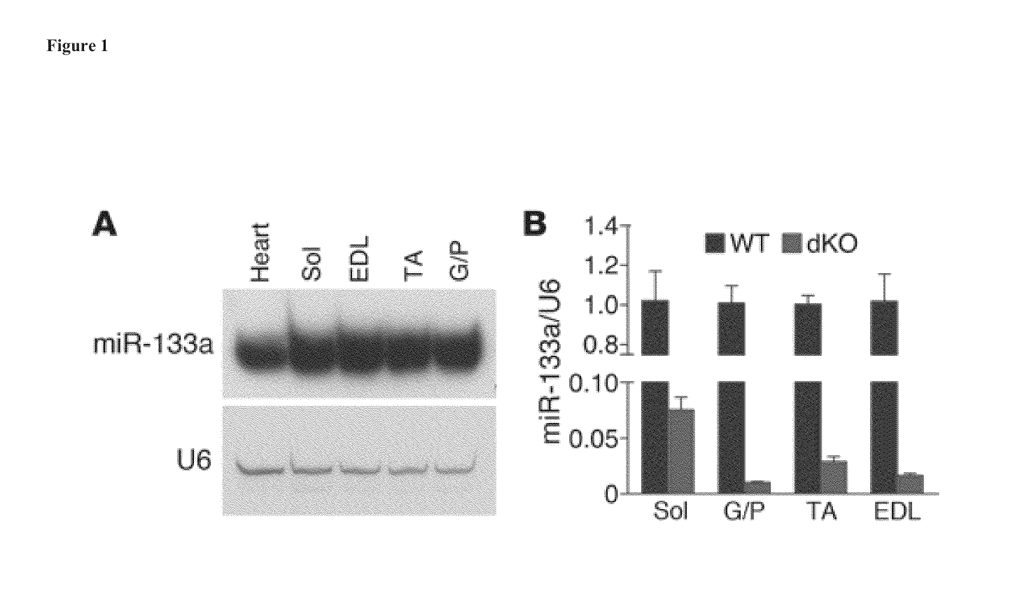

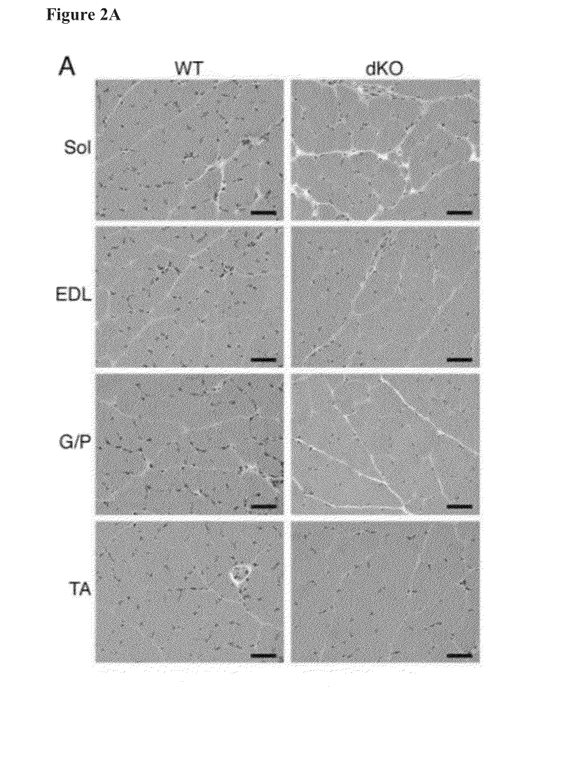

The expression of miR-133 by Northern blot analysis in several skeletal muscles of different myofiber contents was determined. Oxidative, type I (slow-twitch) myofibers are enriched in soleus muscle, and glycolytic type II (fast-twitch) myofibers are enriched in other muscle groups, such as gastrocnemius and plantaris (G/P), tibialis anterior (TA), and extensor digitorum longis (EDL) muscles. miR-133a was expressed at equivalent levels in all of these muscle groups (FIG. 1A), indicative of its comparable levels in type I and type II myofibers. MiR-133b was co-transcribed with miR-206 and was enriched in soleus muscle, which contains predominantly type I fibers (17).

MiR-133a^{-/-} (i.e., dKO) mice by interbreeding miR-133a-1^{+/-}miR-133a2^{+/-} mice were generated, as described previously (18), and the loss of miR-133a expression in dKO skeletal muscle was confirmed by quantitative real-time RT-PCR (FIG. 1B). The low level of miR-133 expression detected in dKO skeletal muscle represented the presence of miR-133b, which is detected by miR-133a probes. Based on results from real-time RT-PCR, the relative abundance of miR-133a versus miR-133b in WT mice was estimated to be about 15:1 in soleus and about 50:1 in G/P, EDL, and TA muscle, which confirms that miR-133b is less abundant than miR-133a in skeletal muscle and is enriched in soleus muscle.

### Example 2

### Accumulation of Centronuclear Myofibers in dKO Skeletal Muscle

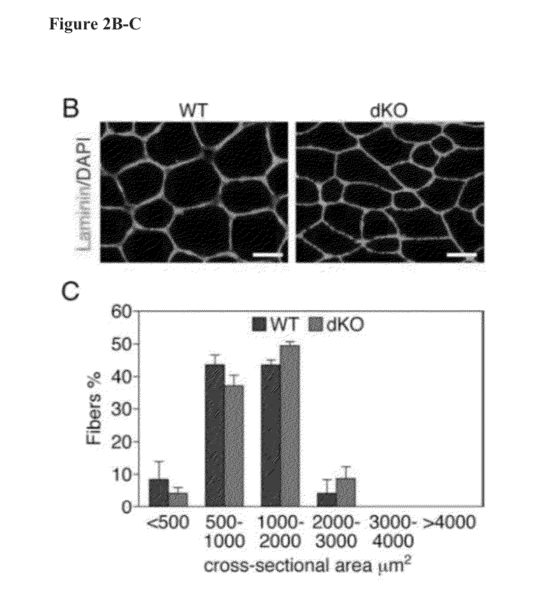

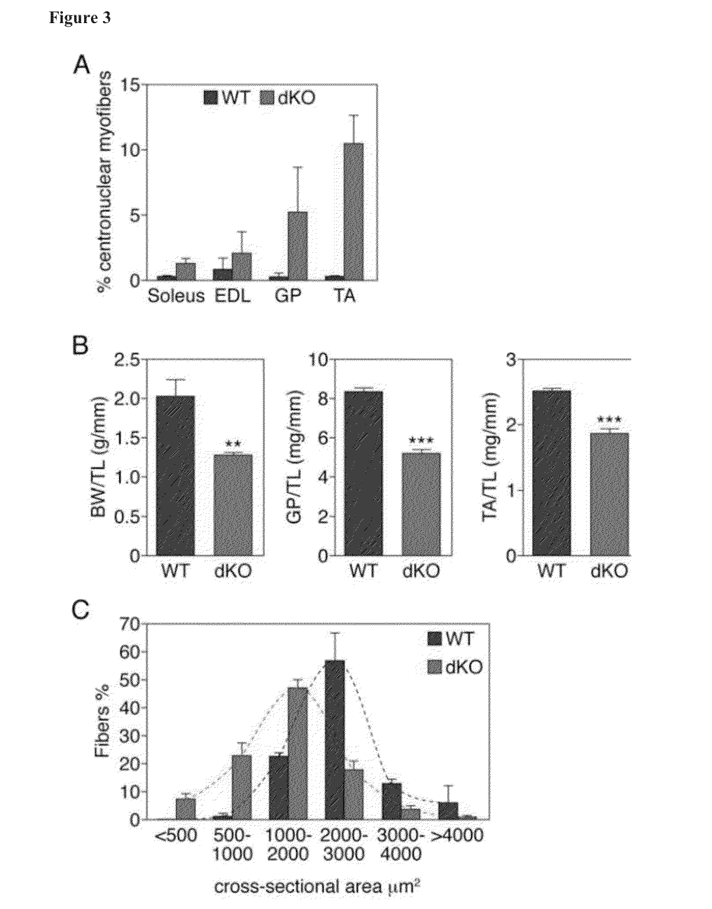

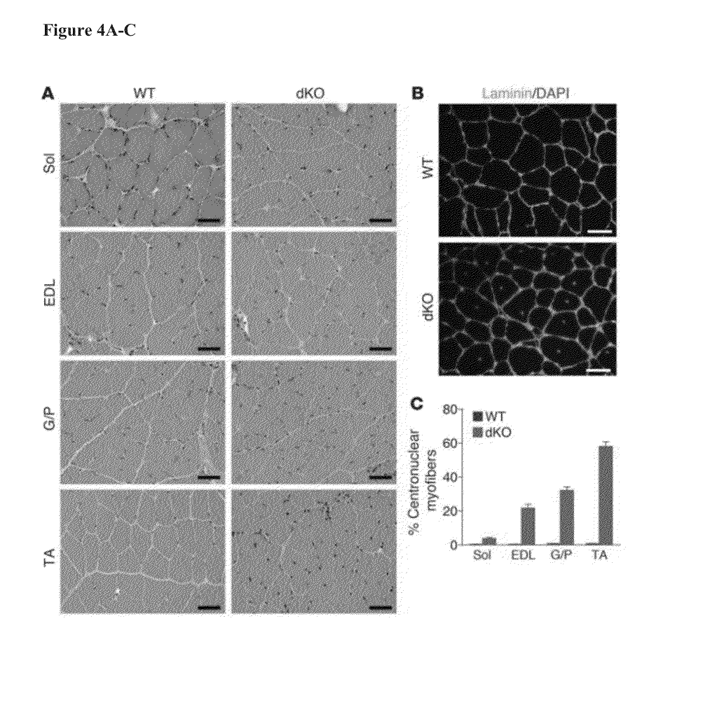

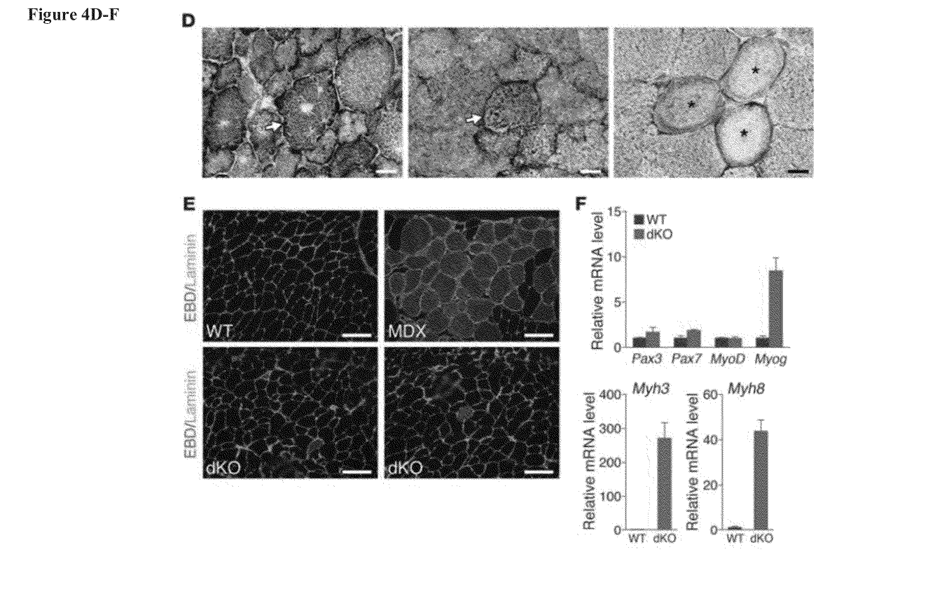

dKO mice did not show apparent abnormalities in mobility. At 4 weeks of age, dKO muscles appeared normal by histological analysis and immunostaining for laminin and DAPI, and myofibers were comparable in size to those of WT muscle (FIG. 2A-C). However, by 6 weeks of age, myofibers with centralized nuclei began to appear in dKO mice, and the percentage of myofibers with central nuclei in EDL, G/P and TA muscle increased progressively with age (FIG. 3A). By 12 weeks of age, nearly 60% of myofibers in TA muscle of dKO mice contained centralized nuclei (FIGS. 4, A-C). In contrast, dKO soleus muscle had relatively few centralized nuclei (FIGS. 4, A and C). These findings suggest that the phenotype of centrally located nuclei in dKO mice is specific to type II myofibers. In addition, at 12 weeks of age, dKO mice were significantly smaller in both body mass and mass of various muscle groups when normalized to tibia length (FIG. 3B). TA myofibers of dKO mice also had smaller diameters than normal at this age (FIG. 3C).

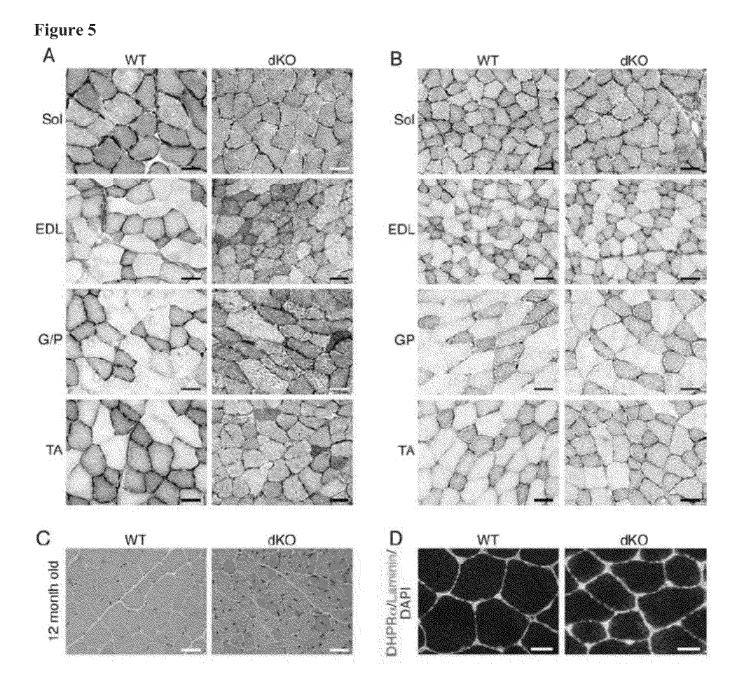

As a further assessment of muscle abnormalities, the distribution of mitochondria and sarcoplasmic reticulum (SR) by NADH-TR staining in dKO muscle fibers at 12 weeks was analyzed. dKO fibers showed more oxidative enzyme activity in G/P, EDL, and TA muscles than did WT myofibers (FIG. 5A), which may reflect a fast-to-slow myofiber conversion (i.e., type II to type I) in these muscles. The oxidative enzyme activity within individual fibers was also unevenly distributed, and some myofibers showed radiating intermyofibrillary networks (FIG. 4D). Ring-like fibers were also occasionally observed upon NADH-TR staining (FIG. 4D). There was no significant difference in NADH-TR staining in soleus muscle between dKO and WT littermates (FIG. 5A). Interestingly, normal NADH-TR staining patterns were observed in 4-week-old dKO muscle when no centrally located nuclei were present (FIG. 5B).

Accumulation of centralized nuclei is usually indicative of muscle regeneration in response to disease or injury (21-23). Thus, signs of muscle damage and degeneration in dKO myofibers at 4 weeks of age were examined. Monitoring sarcolemmal integrity by the uptake of Evans blue dye (EBD), which accumulates in damaged cells, showed very few dye-positive fibers (less than 4 per transverse section) (FIG. 4E). Muscle from mdx mice, which develop muscular dystrophy, were examined for comparison; these mice showed extensive EBD uptake (FIG. 4E). Serum levels of creatine kinase (CK) activity, indicative of sarcolemmal leakage, was analyzed and only slightly elevated (2-fold) CK levels in dKO mice at 3 months (data not shown) was observed. In addition, dKO myofibers showed no signs of inflammation, fibrosis, or apoptosis (data not shown), which are characteristic of dystrophic muscle fibers. At 12 months of age, worsening in myofiber morphologies or signs of inflammation, fibrosis, or cell death in dKO myofibers were not observed (FIG. 5C).

To assay for muscle regeneration, expression of mRNAs encoding several myogenic markers of regeneration was analyzed. Expression of Myog (which encodes myogenin) was upregulated 7-fold in dKO TA muscle, but there was no change in the expression levels of other myogenic markers, such as Pax3, Pax7, and MyoD (FIG. 4F). Although there was a strong increase in both embryonic (Myh3) and perinatal MHC (Mhy8) mRNA levels in TA muscle by real-time RT-PCR (FIG. 4F), embryonic MHC protein was rarely detected in dKO muscle fibers by immunohistochemistry (data not shown). These data indicate that there is only rare muscle regeneration in dKO mice, which is insufficient to account for the extensive centronuclear fibers observed in these mice. Thus, centronuclear myofibers in dKO mice without apparent necrosis, myofiber death, or significant regeneration are pathological characteristics reminiscent of human CNMs (1, 2).

### Example 3

### T-Tubule Disorganization in Muscle Fibers of dKO Mice

In skeletal muscle, excitation-contraction coupling occurs at triads, which are composed of a transverse tubule (T-tubule) and 2 terminal cisternae of the SR (24). In Mtm1-deficient mice, muscle fibers have a decreased number of triads and abnormal organization of T-tubules (3). T-tubule disorganization has also been reported in human CNM patients (6, 25).

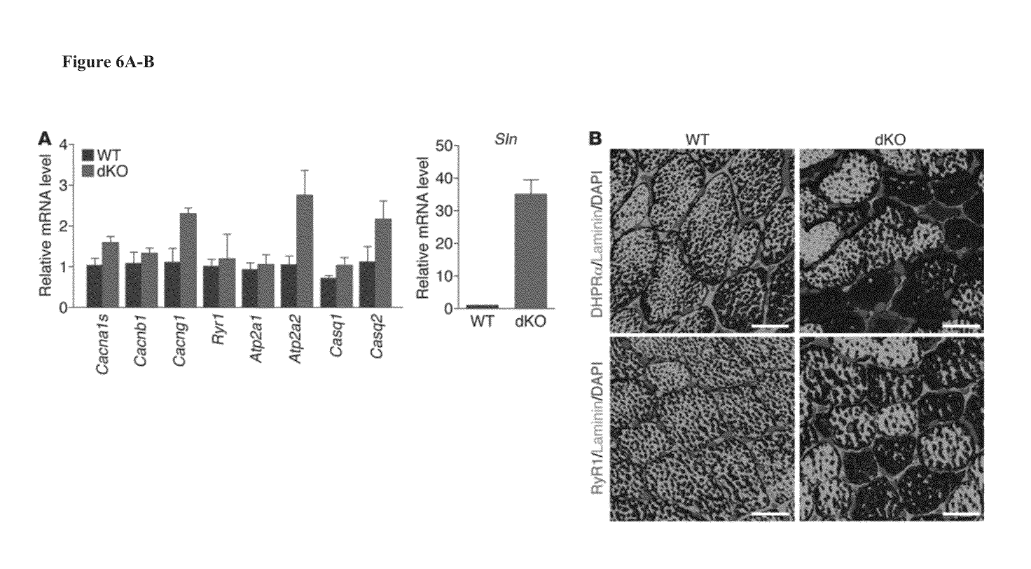

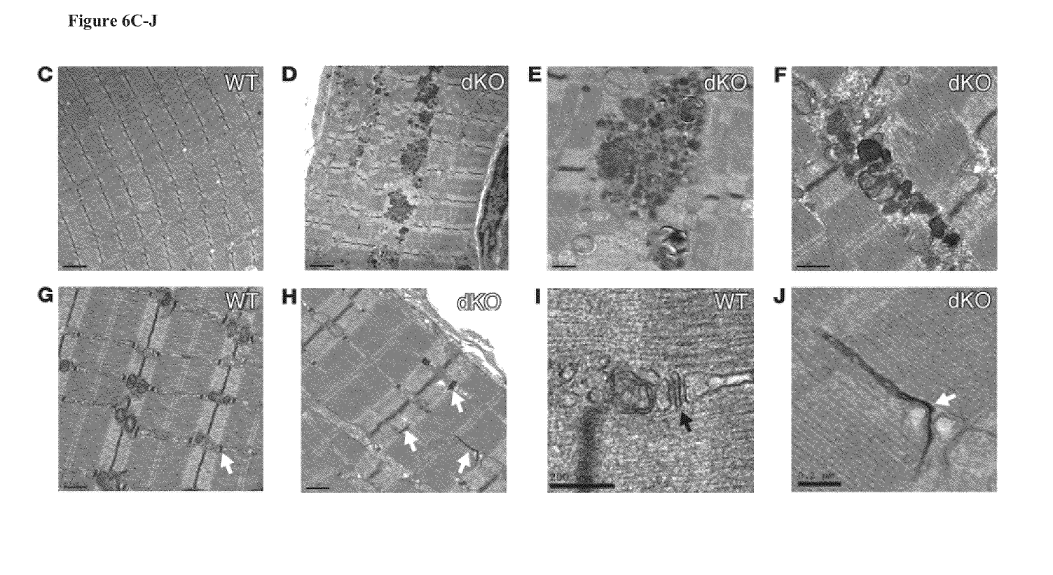

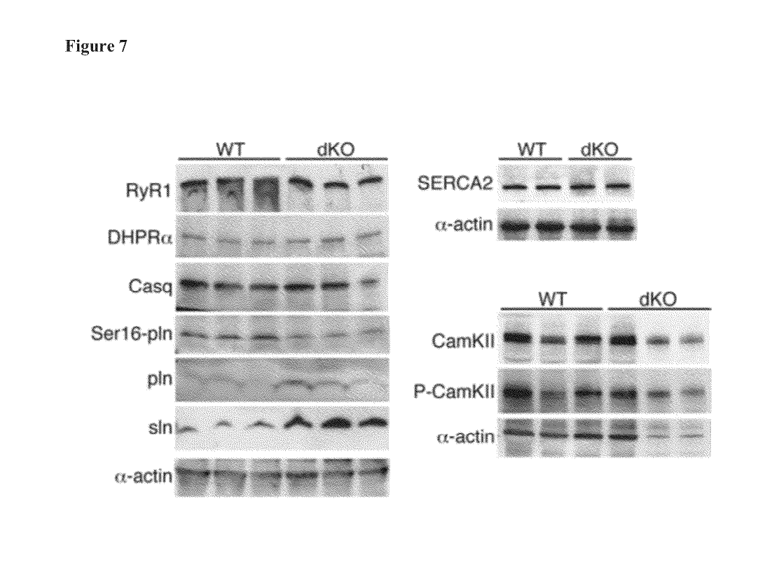

To assess whether T-tubule organization is affected in dKO muscle, the expression of genes encoding components of T-tubules and SR that are important for excitation-contraction coupling, including the α1, β1, and γ1 subunits of the dihydropyridine receptor (DHPR) (encoded by Cacna1s, Cacnb1, and Cacng1, respectively), ryanodine receptor 1 (Ryr1), type 1 and 2 SERCA pumps (Atp2a1 and Atp2a2), and calsequestrin 1 and 2 (Casq1 and Casq2), were examined. At the mRNA level, expression of most of the genes was unchanged, except for a 2.5-fold increase in Cancng1 (FIG. 6A). The expression of RyR1, DHPRα, calsequestrin, and SERCA2 at the protein level was also examined and minimal changes were observed (FIG. 7). In contrast, a 35-fold increase in mRNA levels of Sin, accompanied by a comparable increase in sarcolipin protein was observed (FIG. 6A and FIG. 7). Sarcolipin upregulation is a common feature in skeletal muscle myopathies (26), but the significance of this upregulation is unknown. Expression of phospholamban was slightly upregulated in dKO muscle, but the phosphorylated phospholamban was slightly decreased at the protein level (FIG. 7).

The organization of triads by immunohistochemistry against DHPRα, a marker for T-tubules, and RyR1, a marker for terminal cisternae of SR was also analyzed. In transverse sections of WT myofibers, both T-tubules and terminal cisternae of SR displayed dot-like staining patterns distributed evenly along the myofibers (FIG. 6B), which reflected the transverse orientations of triads relative to sarcomeres. In dKO myofibers, however, both T-tubules and SR showed aggregated staining, absence of staining in some regions, and irregular distribution within individual fibers (FIG. 6B). In addition, in WT muscle, adjacent myofibers showed the same staining patterns. However, in dKO muscle, the adjacent myofibers often displayed different staining patterns (FIG. 6B), suggestive of different orientations of triads in adjacent fibers. At 4 weeks of age, when dKO mice had not yet developed CNM, T-tubule structures were normal, as demonstrated by DHPRα staining (FIG. 5D).

The morphology of triads at the ultrastructural level by electron microscopy (FIG. 6, C-J) was further analyzed. In adult dKO TA muscle fibers, some T-tubules (stained dark by potassium ferricyanide) showed abnormal morphologies and longitudinal orientations aligned with the direction of myofibrils; these were rarely observed in WT muscle fibers (FIG. 6, G-J). Accumulation of electron-dense membranous structures along the myofibers and at triads in dKO myofibers was also observed (FIG. 6, D-F). Overall, these findings indicate that miR-133a is important for the organization of T-tubules and triads and that its absence results in T-tubule disorganization.

### Example 4

### Mitochondrial Dysfunction in dKO Skeletal Muscle

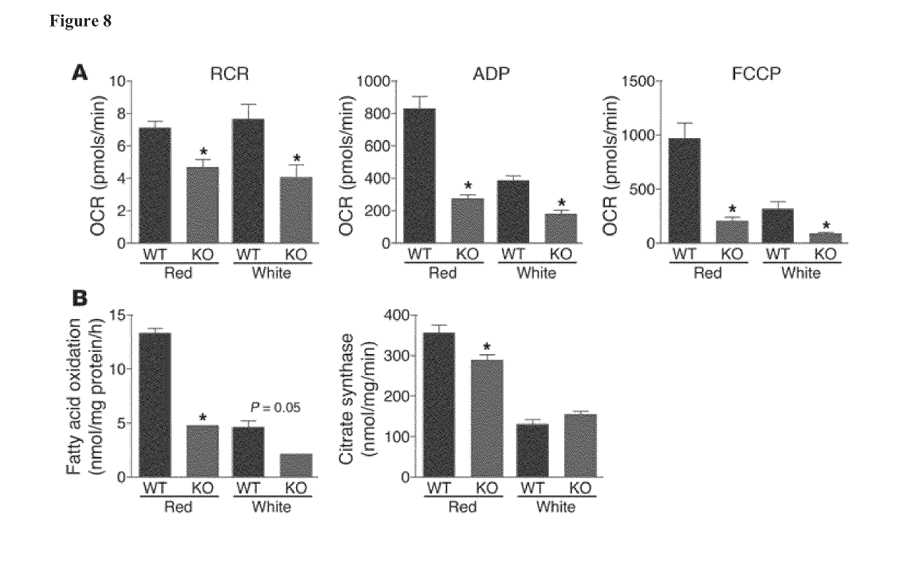

To determine whether lack of miR-133a alters mitochondrial function in skeletal muscle, mitochondria were isolated from red and white portions of the gastrocnemius muscle from dKO and WT mice. Immediately after isolation, mitochondrial respiration and fatty acid oxidation were assessed. Assessments of mitochondrial function include: (a) respiratory control ratio (RCR), the coupling between oxidative phosphorylation and ATP synthesis; (b) ADP-stimulated state 3 respiration, the respiratory rate during which the mitochondria are producing ATP; and (c) carbonylcyanide-p-trifluoromethoxyphenylhydrazone-stimulated (FCCP-stimulated) respiration, the maximal respiratory rate when oxidative phosphorylation is uncoupled from ATP synthesis. A reduction in any of these measures suggests defects in the electron transport chain, Krebs cycle, or ATP synthase activity. The absence of miR-133a resulted in significant declines in RCR, ADP-stimulated state 3 respiration, and FCCP-stimulated maximal respiration in both red and white muscle, although the effects on FCCP-stimulated maximal respiration appeared to be more pronounced in red muscles (FIG. 8A). In addition, total fatty acid oxidation was also significantly lower in mitochondria isolated from both red and white portions of gastrocnemius muscle from dKO animals (FIG. 5B). There was also a reduction in citrate synthase in red quadricep muscle, but not in white quadricep muscle (FIG. 8B). Collectively, these results demonstrate that the absence of miR-133a results in lower intrinsic mitochondrial function and fatty acid oxidation in both red and white skeletal muscle.

### Example 5

### miR-133a Targets Dynamin 2, a Regulator of CNM

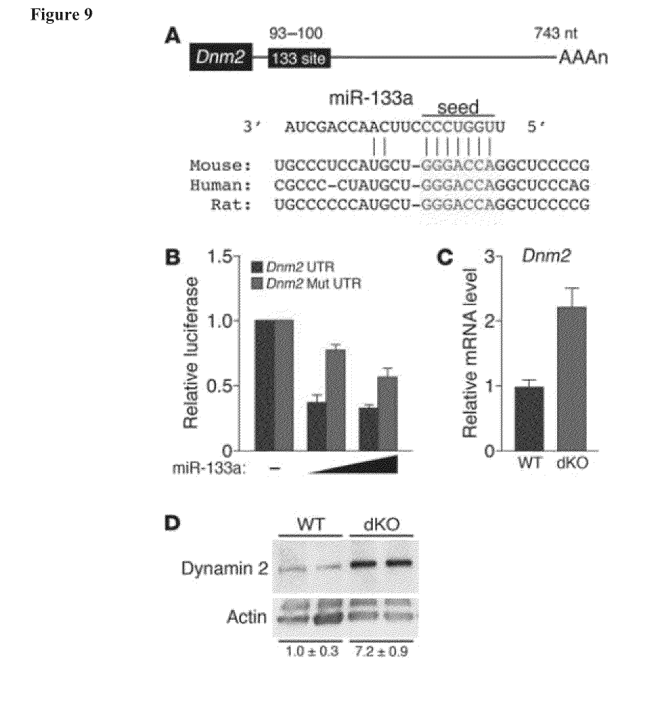

To explore the mechanistic basis of skeletal muscle abnormalities in dKO mice, targets of miR-133a with potential roles in CNM were searched. Among the strongly predicted targets of miR-133a is Dnm2, a large GTPase implicated in endocytosis, membrane trafficking, and regulation of the actin and microtubule cytoskeletons (11). Point mutations in the human DNM2 gene, thought to act in a dominant-negative manner, cause the autosomal-dominant form of CNM (7, 8, 27, 28). The 3′ UTR of Dnm2 mRNA contains an evolutionarily conserved miR-133a binding site (FIG. 9A). miR-133a repressed a luciferase reporter gene linked to the 3′ UTR of Dnm2 mRNA, whereas a mutation in the predicted miR-133a binding site in the 3′ UTR prevented repression (FIG. 9B), confirming Dnm2 mRNA as a target for miR-133a. Moreover, a 2-fold increase in Dnm2 mRNA by quantitative real-time RT-PCR and an approximate 7-fold increase in dynamin 2 protein in TA muscle of dKO compared with WT mice by Western blot analysis was observed (FIGS. 9, C and D). These results indicate that miR-133 represses dynamin 2 expression at both mRNA and protein levels.

### Example 6

### Overexpression of Dynamin 2 in Skeletal Muscle Causes CNM in Type II Myofibers

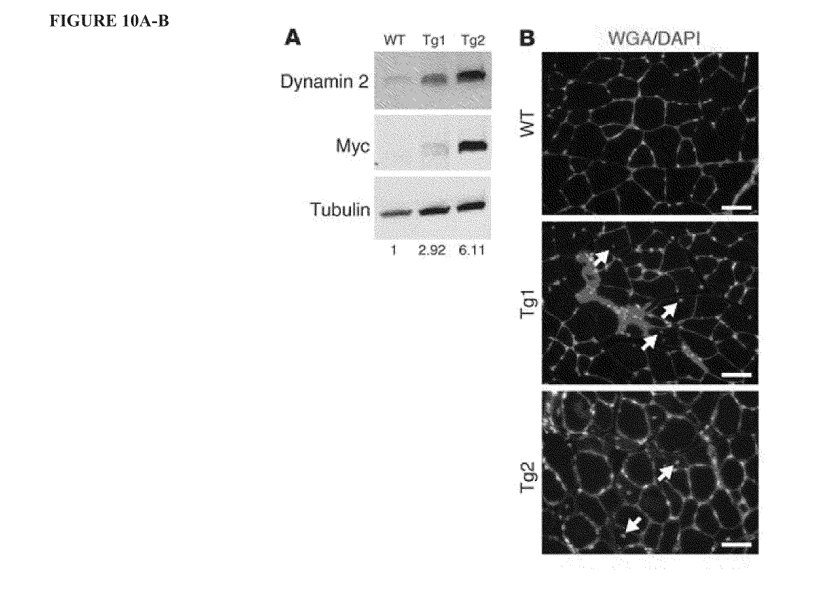

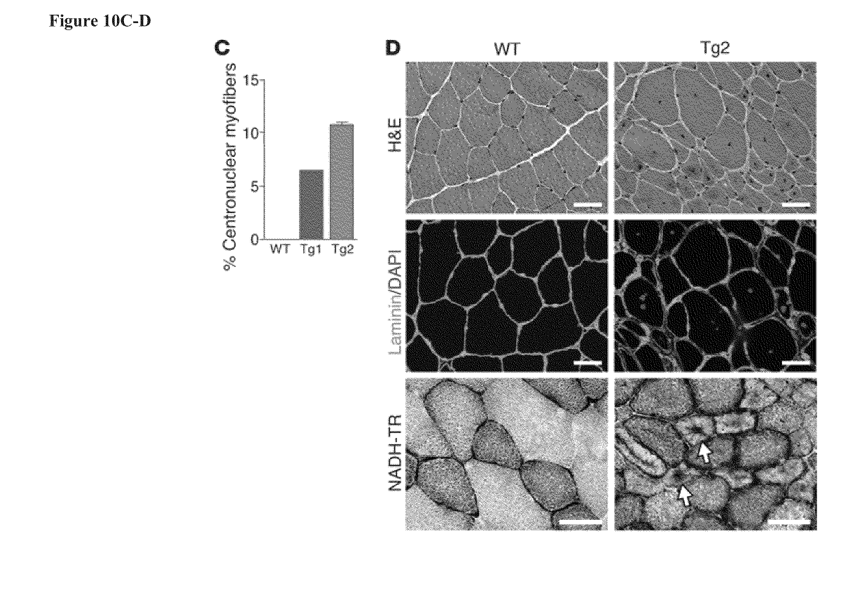

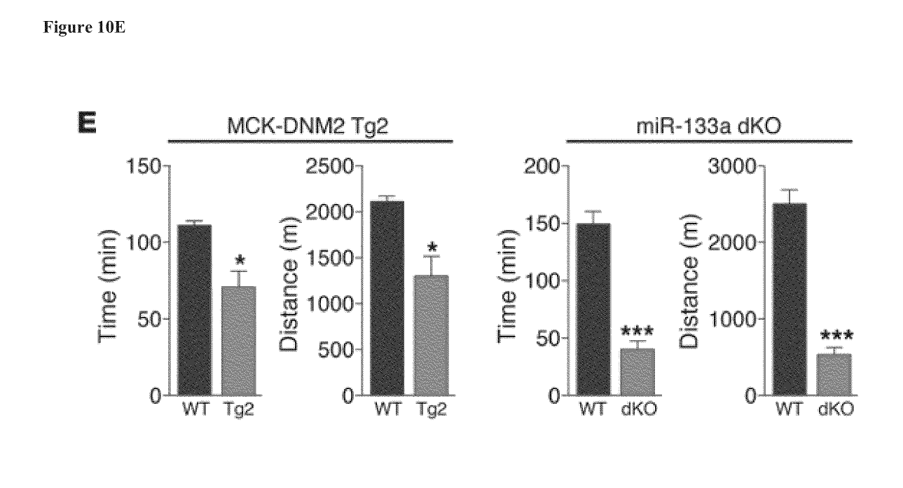

To examine whether elevated expression of dynamin 2, as observed in dKO myofibers, is sufficient to cause CNM, transgenic mice in which dynamin 2 protein (with a myc-tag on the C terminus) was expressed under control of the muscle CK (MCK) promoter (referred to herein as MCK-DYN2 mice) (29, 30) were generated. Overexpression of dynamin 2 protein in skeletal muscle of transgenic mice was confirmed by Western blotting using antibodies against dynamin 2 as well as the myc epitope tag (FIG. 10A). Two MCK-DYN2 transgenic mouse lines, Tg1 and Tg2, which showed 3- and 6-fold overexpression of dynamin 2, respectively, compared with WT levels, was observed. At 7 weeks of age, both transgenic lines displayed accumulation of centronuclear myofibers (FIG. 10B). Interestingly, Tg2 mice, which overexpressed dynamin 2 at a level similar to that of dKO mice, displayed age-dependent centronuclear myofibers in TA muscle comparable to those of dKO mice (FIG. 10C).

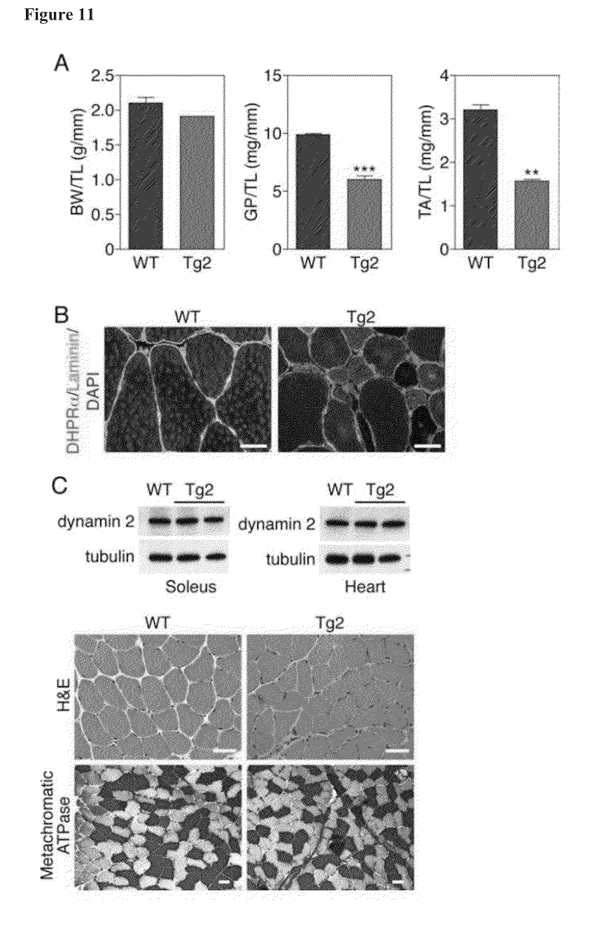

At 11 weeks of age, Tg2 mice displayed signs of muscle atrophy, with decreased muscle mass in both TA and G/P muscle (FIG. 11A). There was no difference in body mass between Tg2 and WT littermates (FIG. 11A). Histological analysis of TA muscle showed heterogeneous fiber sizes and the presence of centronuclear fibers in Tg2 mice (FIG. 10D). The percentage of centronuclear myofibers in TA muscle of Tg2 mice was approximately 23% at this age (data not shown). NADH-TR staining revealed abnormal aggregation of oxidative enzymatic activity and radiating intermyofibrillary networks (FIG. 10D). Abnormal organization of T-tubules was also observed in Tg2 TA muscle, as detected by immunohistochemistry against DHPRα (FIG. 11B).

Dynamin 2 protein was not significantly overexpressed in soleus muscle or heart of Tg2 mice (FIG. 11C), consistent with the preferential expression of the MCK promoter in type II myofibers (29, 30). Not surprisingly, therefore, no abnormalities in soleus muscle or heart function in Tg2 mice was observed (FIG. 11C and data not shown).

To assess muscle performance, mice were subjected to downhill treadmill running and analyzed running time and distance to exhaustion. At 10 weeks of age, Tg2 mice ran for a significantly shorter time than did WT mice (FIG. 10E), indicative of muscle weakness. dKO mice showed a more dramatic decrease in running capacity (FIG. 10E). However, the compromised cardiac function in dKO mice may also be a contributing factor to the reduction in exercise capacity.

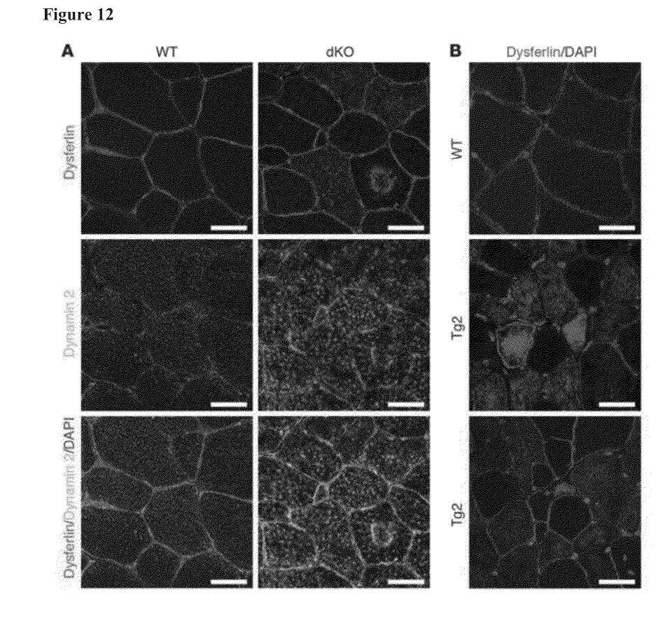

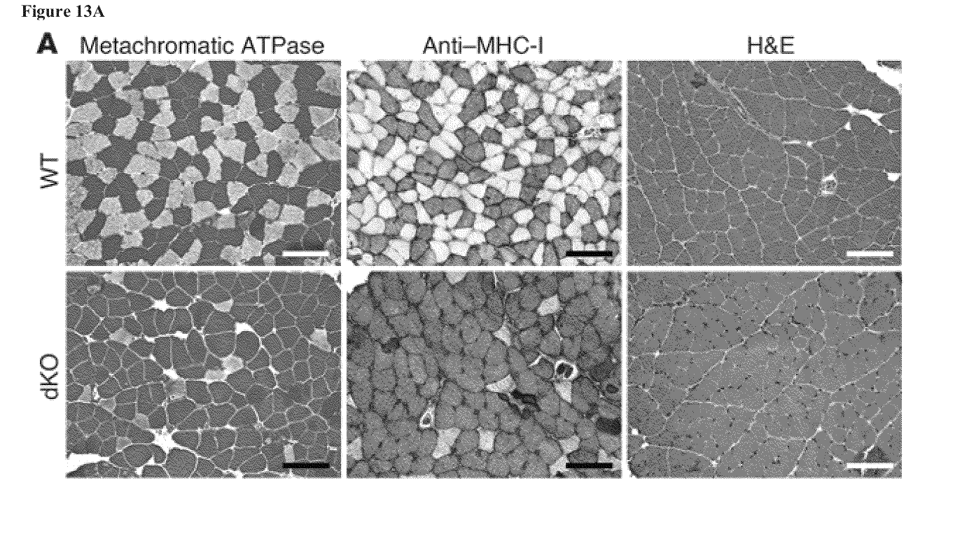

Intracellular accumulation of dysferlin has been recently reported in human DNM2-associated CNM patients, as well as in heterozygous mice carrying the R456W Dnm2 mutation (9). Localization of dysferlin in dKO muscle and Tg2 muscle was also analyzed. Interestingly, substantial accumulation of dysferlin inside the myofibers was observed in both dKO and Tg2 muscle fibers (FIGS. 12, A and B). Furthermore, at least some of the intracellular dysferlin was colocalized with dynamin 2 in dKO muscle fibers (FIG. 12A).

These results demonstrate that elevated expression of Dnn2 in skeletal muscle causes CNM, predominantly in type II fibers, mimicking the dKO phenotype. Therefore, the CNM in dKO muscle can be explained, at least in part, by dysregulation of Dnm2.

### Example 8

### dKO Mice Show Increased Type I Myofibers in Soleus Muscle

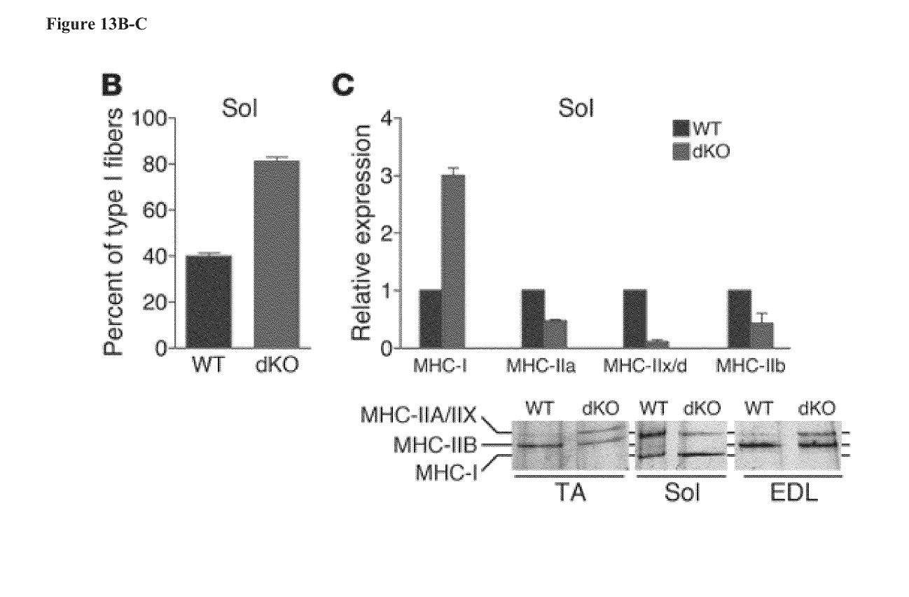

In addition to CNM, dKO mice displayed increased numbers of type I fibers in soleus muscle, which does not show CNM. The fiber type composition of soleus muscle from adult dKO mice was analyzed by metachromatic ATPase staining and by immunohistochemistry against type I myosin heavy chain (MHC), shown by dark brown staining. Soleus muscle of WT mice was composed of about 43% type I fibers (FIGS. 13, A and B). Soleus muscle of dKO mice showed a 2-fold increase in the number of type I fibers (FIGS. 13, A and B).

Quantitative real-time RT-PCR analysis of the expression of transcripts encoding individual MHC isoforms revealed an increase in type I MHC (MHC-I) and decreases in type II MHCs (MHC-IIa, MHC-IIx/d, and MHC-IIb) in soleus muscle of dKO compared with WT mice (FIG. 13C). The protein composition of MHC isoforms in soleus, EDL, and TA muscle was examined by silver staining of glycerol gels: 3 bands were present in protein extracts of soleus muscle isolated from WT mice, corresponding to MHCIIa/IIx, MHC-IIb, and MHC-I proteins; 2 bands were present in protein extracts of TA and EDL muscles from WT mice, representing MHC-IIb and MHC-IIa/IIx (FIG. 13C). Consistent with results from quantitative real-time RT-PCR, soleus muscle of dKO mice displayed an increase in MHC-I protein and a decrease in MHCIIa/IIx proteins. MHC-IIb protein was not observed in dKO soleus muscle. Interestingly, there was an increase in the oxidative MHCIIa/IIx protein and a decrease in the glycolytic MHC-IIb protein in TA and EDL muscles of dKO mice compared with WT mice, which indicates that these muscle groups also display a fiber type shift toward more oxidative (type IIa) fibers.

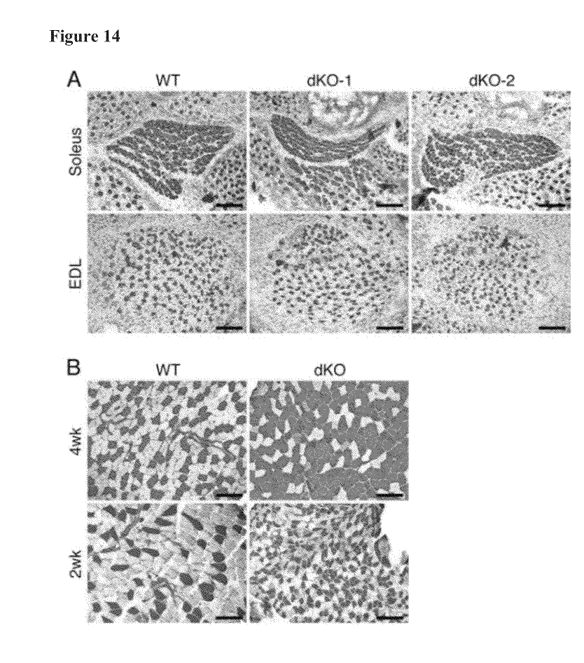

To determine whether loss of miR-133a affects the formation of type I fibers during fetal development, MHC-I expression was examined by immunohistochemistry at P1. There was no obvious difference in the number of MHC-I-positive myofibers in soleus or EDL muscles of dKO mice at P1 (FIG. 14A), which indicates that miR-133a does not influence embryonic development of type I myofibers. To determine when the fiber type switch takes place in dKO mice, fiber type composition in both 2- and 4-week old mice was analyzed by metachromatic ATPase staining. At both ages, the percentage of type I fibers in soleus was increased by almost 2-fold in dKO mice (FIG. 14B). Therefore, miR-133a does not influence specification of type I myofibers during embryonic development. Rather, miR-133a represses type I myofibers postnatally, such that the absence of miR-133a results in an increase in type I myofibers of adult mice.

The examples show that adult mice lacking miR-133a developed progressive CNM, accompanied by mitochondrial dysfunction and fast-to-slow myofiber conversion. Thus, the absence of miR-133a resulted in CNM, mitochondrial dysfunction, disarray of muscle triads, and fast-to-slow myofiber conversion (type II to type I). These muscle abnormalities can be attributed, at least in part, to upregulation of dynamin 2, a target for repression by miR-133a-1 and miR-133a-2. Thus, the findings illustrate the essential role for miR-133a in the maintenance of adult skeletal muscle structure and function and as a modulator of CNMs. MiR-133a has a role in maintaining normal structure and function of adult skeletal muscle.

The skeletal muscle abnormalities in dKO mice were remarkably similar to those of human CNMs, indicative of an important role of this miRNA in modulation of this disorder. The histological features of dKO muscle, including the presence of centronuclear fibers and absence of necrosis or myofiber death, demonstrated similarities to human CNMs. NADH-TR staining patterns in dKO fibers mimicked the typical NADH-TR staining pattern of DNM-associated CNM, which shows radial distribution of sarcoplasmic strands (2). However, in contrast to human CNMs, centronuclear fibers were observed in type II fibers and not in type I fibers in dKO mice. Loss of miR-133a in mouse skeletal muscle caused CNM only in type II fibers, in contrast to the type I fiber predominance in human DNM2-associated CNM patients. It is likely that, in mice, the soleus muscle is protected from muscle damage. However, we cannot rule out the possibility that the soleus-enriched miR-133b, which is highly homologous to miR-133a, protects soleus muscle from CNM. The lack of CNM phenotype in soleus muscle of dKO mice could be due to the expression of miR-133b, which was enriched in soleus muscle. Alternatively, the differences in myofiber distribution of centralized nuclei between mice and humans may reflect species differences in muscle function.

The inventors previously reported type II fiber-specific CNM in mice lacking the Srpk3 gene, which encodes a muscle-specific serine, arginine protein kinase (SRPK) regulated by MEF2 (31). Given the histological similarities between skeletal muscle of Srpk3-null mice and the dKO mice of the present study, it is possible that miR-133a and Srpk3 act through common mechanisms to influence muscle structure and function.

Multiple missense mutations within the DNM2 gene have been linked to autosomal-dominant CNMs (7, 8, 27, 28). Interestingly, these mutations are heterozygous missense mutations or small deletions that do not affect DNM2 transcript levels, protein expression, or localization (8, 28). However, the mechanisms whereby CNM-associated mutations affect DNM2 cellular function are unknown.

The examples demonstrate that miR-133a directly regulated Dnm2 mRNA and dynamin 2 protein expression. Moreover, elevated expression of Dnm2 in skeletal muscle, at levels comparable to those in dKO mice, caused CNM, which indicates that skeletal muscle function depends on a precise level of DNM2 expression. Although the exact mechanism is unknown, it is possible that increased dynamin 2 protein may cause abnormally strong dynamin assembly and disrupt the energetic balance of efficient assembly and disassembly that is required for proper DNM2 function in skeletal muscle. In this regard, CNM-related DNM2 mutations in humans have been reported to act in a dominant-negative manner to impair membrane trafficking, cytoskeleton-related processes, and centrosomal function (8, 28).

It is unclear how Dnm2 gain of function in dKO mice and MCK-DNM2 transgenic mice also cause CNM. However, a recent study showed that specific CNM-related DNM2 mutations cause increased GTPase activity and promotes dynamin oligomerization without altering lipid binding (32). Another study also showed that CNM-related DNM2 mutants enhance the stability of dynamin polymers without impairing their ability to bind and/or hydrolyze GTP (33). In another study, heterozygous mice expressing the most frequent Dnm2 mutation, R456W, developed a myopathy with muscle atrophy and weakness, but not CNM (9). It was suggested that the effect of dynamin 2 on contractile properties and nuclear positioning are independent. Intriguingly, the examples demonstrated that overexpression of Dnm2 in skeletal muscle affected both muscle function and nuclear position. The difference in these phenotypes could be explained by different model systems used (i.e., overexpression vs. knockin). Nonetheless, the examples demonstrated that skeletal muscle is sensitive to dynamin 2 protein level and that elevated dynamin 2 expression results in CNM in mice.

miR-133a is also predicted to target other genes, such as those encoding profilin 2, calmodulin 1, FGFR1, and mastermind-like 1. Luciferase reporter assays with the 3′ UTRs of these mRNAs and confirmed that they were targeted by miR-133a in vitro; however, their regulation by miR-133a in vivo was less prominent in skeletal muscle (data not shown). Therefore, although miR-133a targets multiple genes in skeletal muscle, the primary effect comes from its regulation of DNM2.

Skeletal muscle is composed of heterogeneous myofibers with distinctive contractile and metabolic properties (34). Adult myofibers are highly plastic and can switch between type I and type II phenotypes in response to work load, hormonal stimuli, and disease. The phenotype of dKO mice indicates that miR-133a suppresses the type I myofiber gene program. Type I myofibers are believed to be more resistant to disease or damage than type II fibers (35). In many muscle diseases, such as Duchenne muscular dystrophy, there is a switch in fiber type toward type I, which may serve as a protective mechanism (36, 37). It may be possible that changes in fiber types in dKO muscle are secondary to the CNM phenotype.

Mitochondrial dysfunction has been implicated in a number of myopathies, including Duchenne muscular dystrophy and metabolic and neurological disorders (38-40), as well as in the aging process (41, 42). The results from the examples are consistent with the previous finding that mitochondrial abnormalities are associated with DNM2-related CNM (43). However, this result may appear to be incompatible with the fast-to-slow myofiber conversion in dKO mice, since type I fibers are believed to have more oxidative enzyme activity. However, the exact mechanism underlying this discrepancy is unclear and there are several possible explanations. The switch to type I fiber could be the result of changes in myosin composition that do not affect mitochondria content. In addition, a fast-to-slow myofiber conversion is associated with increases in capillary and mitochondrial density. This does not take into account the functional capacity of the individual mitochondria. Finally, impairments in mitochondrial function result in reduced ATP availability to the muscle. Thus, it is possible that the fiber type switch in dKO muscle is a protective mechanism against mitochondrial dysfunction and reduced ATP availability (35).

The results from the examples demonstrated that miR-133a, which is expressed in both heart and skeletal muscle, plays different roles in these tissues. In the heart, miR-133a regulates cardiomyocyte proliferation and suppresses smooth muscle gene program during heart development (18). miR-133a was dispensable for skeletal muscle development, as dKO mice did not display any skeletal muscle abnormalities until after 4 weeks of age. The skeletal muscle of dKO mice developed CNM after 4 weeks of age, whereas the heart develops dilated cardiomyopathy at a later age, which leads to heart failure and sudden death in a subset of mice (18). Interestingly, the hearts of dKO mice showed pronounced sarcomere disorganization and disrupted Z-discs, as well as severe mitochondrial abnormalities at 4 months of age (18). On the other hand, sarcomeric structures and mitochondrial morphologies were largely unaffected in dKO skeletal muscle (FIG. 3). Rather, miR-133a specifically affects triads in skeletal myofibers. It is unclear why the heart and skeletal muscle show different abnormalities in response to the loss of miR-133a. It may reflect the regulation of different target genes by miR-133a in skeletal muscle (such as dynamin 2) and heart (such as cyclin D2 and SRF). Another reason could be the fact that the highly homologous miR-133b is expressed in dKO skeletal muscle, albeit at a lower level, but not in dKO heart. Although it is conceivable that the cardiomyopathy in dKO mice could contribute, CNM is not associated with other mouse models of cardiomyopathy. Therefore, the results indicate that the skeletal muscle abnormalities in dKO mice regulated by miR-133a are believed to be mainly caused by cell-autonomous functions of miR-133a in skeletal muscle.

The similarities in skeletal muscle abnormalities in dKO mice and human CNM patients suggest that miR-133a plays a modulatory role in human myopathies. In this regard, the present invention provides compositions and methods for modulating miR-133a mRNA targets, such as DNM2, by administering a miR-133a agonist, such as a miR-133a polynucleotide.

### Methods

Generation of MCK-DNM2 Transgenic Mice.

A MCK-DNM2 transgene was generated by placing a C-terminal myc-tagged rat Dnm2 cDNA (gift from J. Albanesi, University of Texas Southwestern Medical Center, Dallas, Tex., USA) downstream of the 4.8-kb MCK promoter. The construct contained a downstream human growth hormone poly(A) signal. Transgenic mice were generated as previously described (44, 45). Two FI lines, termed Tg1 and Tg2, were analyzed.

Northern Blot Analysis.

Total RNA was isolated from mouse skeletal muscle tissues using the miRNeasy mini kit (QIAGEN). Northern blots to detect miR-133a and U6 were performed as described previously (18). ³²P-labeled Star-Fire oligonucleotide probes (IDT) against mature miR-133a and U6 probes were used in the hybridization.

### RT-PCR and Real-Time Analysis.

RNA was treated with Turbo RNase-free DNase (Ambion Inc.) prior to the reverse transcription step. RT-PCR was performed using random hexamer primers (Invitrogen). Quantitative real-time RT-PCR was performed using TaqMan probes (ABI) or Sybr Green probes. Sybr Green primers used in FIG. 6 (as described in as described (3)):

Cacna1s For primer:
(SEQ ID NO: 5)
5′-tccagct actgccatgctgat-3′
Cacna1s Rev primer
(SEQ ID NO: 6)
5′-tcgacttcctctggttccat-3′
Cacnb1 For primer
(SEQ ID NO: 7)
5′-ctttgcctttgagctagacc-3′
Cacnb1 Rev primer
(SEQ ID NO: 8)
5′-gcacgtgctctgtcttctta-3
Cacng1 For primer
(SEQ ID NO: 9)
5′-catctgcgcatttctgtcct-3′
Cacng1 Rev primer
(SEQ ID NO: 10)
5′-atcat acgcttcaccgactg-3′
Ryr1 For primer
(SEQ ID NO: 11)
5′-gtt atcgtcattctgctggc-3′
Ryr1 Rev primer
(SEQ ID NO: 12)
5′-gcctattccacagatgaagc-3′
Atp2a1 For primer
(SEQ ID NO: 13)
5′-tggctcatggtcctcaagat-3′
Atp2a1 Rev primer
(SEQ ID NO: 14)
5′-cctcagctttggctgaagat-3′
Atp2a2 For primer
(SEQ ID NO: 15)
5′-agcttggagcaggtcaagaa-3′
Atp2a2 Rev primer
(SEQ ID NO: 16)
5′-gctctacaaaggctgtaatcg-3′
Casq1For primer
(SEQ ID NO: 17)
5′-actcagagaaggatgcagct-3′
Casq1 Rev primer
(SEQ ID NO: 18)
5′-ctctacagggtcttctagga-3′
Casq2 For primer
(SEQ ID NO: 19)
5′-gtgtcttcagacaaggtctc-3′
Casq2 Rev primer
(SEQ ID NO: 20)
5′-acccttcagaacatacaggc-3′

Sybr Green primers used in FIG. 13 (as described in (52)):

MHC-1 For primer
(SEQ ID NO: 21)
5′-CCTTGGCACCAATGTCCCGGCTC-3′
MHC-1 Rev primer
(SEQ ID NO: 22)
5′-GAAGCGCAATGCAGAGTCGGTG-3
MHC-IIa For primer
(SEQ ID NO: 23)
5′-ATGAGGTCCGACGCCGAG-3′
MHC-IIa Rev primer
(SEQ ID NO: 24)
5′-TCTGTTAGCATGAACTGGTAGGCG-3′
MHC-IIx For primer
(SEQ ID NO: 25)
5′-AAGGAGCAGGACACCAGCGCCCA-3′
MHC-IIx Rev primer
(SEQ ID NO: 26)
5′-ATCTCTTTGGTCACTTTCCTGCT-3′
MHC-IIb For primer
(SEQ ID NO: 27)
5′-GTGATTTCTCCTGTCACCTCTC-3′
MHC-IIb Rev primer
(SEQ ID NO: 28)
5′-GGAGGACCGCAAGAACGTGCTGA-3′.

Quantitative real-time RT-PCR on miRNA was performed using the Taq-Man miRNA assay kits (ABI) according to manufacturer's protocol.

### Histological Analysis of Skeletal Muscle.

Various muscle groups were harvested, flash frozen in embedding medium containing a 3:1 mixture of Tissue Freezing Medium (Triangle Biomedical Sciences) and gum tragacanth (Sigma-Aldrich) or fixed in 4% paraformaldehyde, and processed for routine paraffin histology. Frozen sections were cut on a cryotome and stained with H&E as previously described (45). NADH-TR staining on frozen sections was performed according to standard protocol. Metachromatic ATPase staining on frozen sections was performed as described previously (44, 45). To determine the number of myofibers with centralized nuclei, more than 500 myofibers were counted for TA and G/P muscles and more than 300 were counted for soleus and EDL muscles of each mouse. Myofiber cross-sectional area was determined using ImageJ, and more than 200 fibers per muscle section were examined.

Electron Microscopy.

Mice were anesthetized, then transcardially perfused with 0.1M phosphate buffer (pH 7.3) followed by 2.5% glutaraldehyde and 2% paraformaldehyde in 0.1 M sodium cacodylate buffer. TA muscles were dissected and processed for selective staining of T-tubules as described previously (3).

### EBD Uptake.

EBD uptake was performed as described previously (46). Briefly, EBD (10 mg/ml in PBS) was administered to mice intraperitoneally (0.1 ml per 10 g body mass). Mice were subjected to exercise using a running wheel overnight (all mice underwent wheel running), and muscles were harvested approximately 18 hours later. Gastrocnemius and TA muscles were flash frozen in embedding medium. Frozen sections were immunostained with primary antibody rabbit anti-laminin (Sigma-Aldrich, 1:200), followed by secondary antibody Alexa Fluor 488-conjugated goat anti-rabbit IgG (Invitrogen, 1:400). EBD was detected as red autofluorescence using fluorescence microscopy.

### Immunohistochemistry.

Frozen sections were fixed in freshly prepared 4% paraformaldehyde for 20 minutes on ice and were then treated with 0.3% Triton X-100 in PBS at room temperature for 20 minutes. Sections were incubated with mouse IgG blocking solution from the M.O.M. kit (Vector Lab) diluted in 0.01% Triton X-100 in PBS at room temperature for 1 hour. Sections were then incubated with 5% goat serum (Sigma-Aldrich) in M.O.M. protein diluent for 30 minutes. Sections were incubated with primary antibodies diluted in M.O.M. protein diluent at 4° C. overnight. The next morning, slides were washed with PBS and incubated with secondary antibodies diluted in M.O.M. protein diluent at room temperature for 45 minutes. Sections were then washed and mounted with VectoShield Mounting Medium with DAPI. Pictures were taken with a Zeiss confocal microscope. Primary and secondary antibodies were as follows: DHPRα(Thermo Scientific, 1:100), RyR1 (clone34C, Sigma-Aldrich, 1:100), Laminin (Sigma-Aldrich, 1:200), MHC-I (clone NOQ7.5.4D, Sigma-Aldrich, 1:5,000), Dysferlin (Hamlet, Novocastra, 1:40), dynamin 2 (Abcam, 1:400), Alexa Fluor 594-conjugated goat anti-mouse IgG1 (Invitrogen, 1:400), Alexa Fluor 488-conjugated goat anti-rabbit IgG (Invitrogen, 1:400). Wheat germ agglutinin staining was performed as previously described (46). MHC-I (clone NOQ7.5.4D, Sigma-Aldrich, 1:5,000) was used for primary detection of type I myosin, and HRP-conjugated secondary antibody (A8924, Sigma-Aldrich) followed by DAB chromagen reaction (DAKO) were used for detection. Samples were then counterstained with hematoxylin.

### Western Blot Analysis.

Total cell lysates were extracted from skeletal muscle tissues and resolved on SDS-PAGE. Western blotting was performed by standard protocol. Antibodies against dynamin 2 (Santa Cruz Biotechnology, 1:100), c-Myc (Santa Cruz Biotechnology, 1:1,000), DHPRα(Thermo Scientific, 1:100). RyR1 (clone 34C, Sigma-Aldrich, 1:100), SERCA2 (BD Biosciences, 1:1,000), sarcolipin (gift from M. Periasamy, Ohio State University, Columbus, Ohio, USA, 1:1,000), phospholamban (Upstate, 1:1,000), phospho-phospholamban (Millipore, 1:1,000), calsequestrin 2 (Santa Cruz, 1:1,000), tubulin (Sigma-Aldrich, 1:5,000) and α-actin (Sigma-Aldrich, 1:2,000) were used. Quantification of Western blots was performed by densitometry using a Phospholmager.

### Cell Culture, Transfection, and Luciferase Assays.

1-kb fragments of the Dnm2 3′ UTR containing the miR-133a binding sites were cloned into pMIRREPORT vector (Ambion). Mutagenesis of the miR-133a binding site, cell culture, and luciferase assay were performed as previously described (18).

### Treadmill Test.

The treadmill test was performed using the Exer-6M (Columbus Instruments) at 15° downhill. Mice were trained on the treadmill at 5 m/min for 5 minutes for 2 consecutive days. The following day, mice ran on the treadmill at 5 m/min for 2 minutes, 7 m/min for 2 minutes, 8 m/min for 2 minutes, and 10 m/min for 5 minutes. Subsequently, speed was increased by 1 m/min to a final speed of 20 m/min. Exhaustion was defined as the inability of the animal to remain on the treadmill despite electrical prodding.

### Electrophoresis of MHC Isoforms.

Myosin was isolated from skeletal muscle and was separated by electrophoresis on glycerol-SDS-PAGE gels as previously described (47). Gels were stained with a silver nitrate staining kit (Bio-Rad).

Mitochondrial Isolation from Gastrocnemius Muscle.

Mitochondria were isolated from red and white skeletal muscle dissected from gastrocnemius muscle as previously described (48), with modifications. Tissue samples were collected in buffer containing 67 mM sucrose, 50 mM Tris/HCl, 50 mM KCl, 10 mM EDTA/Tris, and 10% bovine serum albumin. Samples were minced and digested in 0.05% trypsin for 30 minutes. Samples were then homogenized, and mitochondria were isolated by differential centrifugation.

### Respiration in Isolated Mitochondria.

Respirometry of isolated mitochondria was performed using an XF24 extracellular flux analyzer (Seahorse Bioscience). Immediately after isolation and protein quantification, mitochondria were plated on Seahorse cell culture plates at 5 μg/well in the presence of 10 mM pyruvate and 5 mM malate. Experiments consisted of 25-second mixing and 4- to 7-minute measurement cycles. Oxygen consumption was measured under basal conditions, ADP-stimulated (5 mM) state 3 respiration, oligomycin-induced (2 μM) state 4 respiration, and uncoupled respiration in the presence of FCCP (0.3 μM) to assess maximal oxidative capacity. The RCR was calculated as the ratio of state 3/state 4 respiration. All experiments were performed at 37° C.

### Fatty Acid Metabolism.

Fatty acid oxidation was assessed in isolated mitochondria by measuring and summing ¹⁴CO₂ production and ¹⁴C-labeled acid-soluble metabolites from the oxidation of [1-¹⁴C]-palmitic acid as previously described (49, 50). Citrate synthase activity was determined as previously described (51).

### Animal Care.

All animal experimental procedures were reviewed and approved by the Institutional Animal Care and Use Committees of University of Texas Southwestern Medical Center.

### Statistics.

Data are presented as mean±SEM. Differences between groups were tested for statistical significance using the unpaired 2-tailed Student's t test. P values less than 0.05 were considered significant.

### REFERENCES

1. Jungbluth H, Wallgren-Pettersson C, Laporte J. Centronuclear (myotubular) myopathy. Orphanet J Rare Dis. 2008; 3:26.
2. Romero N B. Centronuclear myopathies: a widening concept. Neuromuscul Disord. 2010; 20(4):223-228.
3. Al-Qusairi L, et al. T-tubule disorganization and defective excitation-contraction coupling in muscle fibers lacking myotubularin lipid phosphatase. Proc Natl Acad Sci USA. 2009; 106(44):18763-18768.
4. Buj-Bello A, et al. AAV-mediated intramuscular delivery of myotubularin corrects the myotubular myopathy phenotype in targeted murine muscle and suggests a function in plasma membrane homeostasis. Hum Mol. Genet. 2008; 17(14):2132-2143.
5. Buj-Bello A, et al. The lipid phosphatase myotubularin is essential for skeletal muscle maintenance but not for myogenesis in mice. Proc Natl Acad Sci USA. 2002; 99(23):15060-15065.
6. Dowling J J, et al. Loss of myotubularin function results in T-tubule disorganization in zebrafish and human myotubular myopathy. PLoS Genet. 2009; 5(2):e1000372.
7. Bitoun M, et al. Dynamin 2 mutations cause sporadic centronuclear myopathy with neonatal onset. Ann Neurol. 2007; 62(6):666-670.
8. Bitoun M. et al. Mutations in dynamin 2 cause dominant centronuclear myopathy. Nat. Genet. 2005; 37(11):1207-1209.
9. Durieux A C, et al. A centronuclear myopathy-dynamin 2 mutation impairs skeletal muscle structure and function in mice. Hum Mol. Genet. 2010; 19(24):4820-4836.
10. Heymann J A, Hinshaw J E. Dynamins at a glance. J Cell Sci. 2009; 122(Pt 19):3427-3431.
11. Durieux A C, Prudhon B, Guicheney P, Bitoun M. Dynamin 2 and human diseases. J Mol Med. 2010; 88(4):339-350.
12. Filipowicz W, Bhattacharyya S N, Sonenberg N. Mechanisms of post-transcriptional regulation by microRNAs: are the answers in sight?Nat Rev Genet. 2008; 9(2): 102-114.
13. Williams A H, Liu N, van Rooij E, Olson E N. MicroRNA control of muscle development and disease. Curr Opin Cell Biol. 2009; 21(3):461-469.
14. Eisenberg I, Alexander M S, Kunkel L M. miRNAS in normal and diseased skeletal muscle. J Cell Mol. Med. 2009; 13(1):2-11.
15. Eisenberg I, et al. Distinctive patterns of microRNA expression in primary muscular disorders. Proc Natl Acad Sci USA. 2007; 104(43):17016-17021.
16. Liu N, Olson E N. MicroRNA regulatory networks in cardiovascular development. Dev Cell. 2010; 18(4):510-525.
17. Williams A H, et al. MicroRNA-206 delays ALS progression and promotes regeneration of neuromuscular synapses in mice. Science. 2009; 326(5959):1549-1554.
18. Liu N, et al. microRNA-133a regulates cardiomyocyte proliferation and suppresses smooth muscle gene expression in the heart. Genes Dev. 2008; 22(23):3242-3254.
19. Zhao Y, et al. Dysregulation of cardiogenesis, cardiac conduction, and cell cycle in mice lacking miRNA-1-2. Cell. 2007; 129(2):303-317.
20. Chen J F, et al. The role of microRNA-1 and microRNA-133 in skeletal muscle proliferation and differentiation. Nat. Genet. 2006; 38(2):228-233.
21. Shi X, Garry D J. Muscle stem cells in development, regeneration, and disease. Genes Dev. 2006; 20(13):1692-1708.
22. McNally E M, Pytel P. Muscle diseases: the muscular dystrophies. Annu Rev Pathol. 2007; 2:87-109.
23. Tedesco F S, Dellavalle A, Diaz-Manera J, Messina G, Cossu G. Repairing skeletal muscle: regenerative potential of skeletal muscle stem cells. J Clin Invest. 2010; 120(1): 11-19.
24. Franzini-Armstrong C, Jorgensen A O, Structure and development of E-C coupling units in skeletal muscle. Annu Rev Physiol. 1994; 56:509-534.
25. Toussaint A, et al. Defects in amphiphysin 2 (BIN 1) and triads in several forms of centronuclear myopathies. Acta Neuropathol. 2011; 121(2):253-266.
26. Babu G J, et al. Ablation of sarcolipin enhances sarcoplasmic reticulum calcium transport and atrial contractility. Proc Natl Acad Sci USA. 2007; 104(45):17867-17872.
27. Bitoun M, et al. A new centronuclear myopathy phenotype due to a novel dynamin 2 mutation. Neurology. 2009; 72(1):93-95.
28. Bitoun M, et al. Dynamin 2 mutations associated with human diseases impair clathrin-mediated receptor endocytosis. Hum Mutat. 2009; 30(10): 1419-1427.
29. Donoviel D B, Shield M A, Buskin J N, Haugen H S, Clegg C H, Hauschka S D. Analysis of muscle creatine kinase gene regulatory elements in skeletal and cardiac muscles of transgenic mice. Mol Cell Biol. 1996; 16(4):1649-1658.
30. Shield M A, Haugen H S, Clegg C H, Hauschka S D. E-box sites and a proximal regulatory region of the muscle creatine kinase gene differentially regulate expression in diverse skeletal muscles and cardiac muscle of transgenic mice. Mol Cell Biol. 1996; 16(9):5058-5068.
31. Nakagawa O, et al. Centronuclear myopathy in mice lacking a novel muscle-specific protein kinase transcriptionally regulated by MEF2. Genes Dev. 2005; 19(17):2066-2077.
32. Kenniston J A, Lemmon M A. Dynamin GTPase regulation is altered by PH domain mutations found in centronuclear myopathy patients. EMBO J. 2010; 29(18):3054-3067.
33. Wang L, Barylko B, Byers C, Ross J A. Jameson D M, Albanesi J P. Dynamin 2 mutants linked to centronuclear myopathies form abnormally stable polymers. J Biol. Chem. 2010; 285(30):22753-22757.
34. Bassel-Duby R, Olson E N. Signaling pathways in skeletal muscle remodeling. Annu Rev Biochem. 2006; 75:19-37.
35. Webster C, Silberstein L, Hays A P, Blau H M. Fast muscle fibers are preferentially affected in Duchenne muscular dystrophy. Cell. 1988; 52(4):503-513.
36. Wang J F, Forst J, Schroder S, Schroder J M. Correlation of muscle fiber type measurements with clinical and molecular genetic data in Duchenne muscular dystrophy. Neuromuscul Disord. 1999; 9(3):150-158.
37. Cros D, Harnden P, Pellissier J F, Serratrice G. Muscle hypertrophy in Duchenne muscular dystrophy. A pathological and morphometric study. J Neurol. 1989; 236(1):43-47.
38. Naumann M, et al. Mitochondrial dysfunction in adult-onset myopathies with structural abnormalities. Acta Neuropathol. 1995; 89(2): 152-157.
39. Kim J A, Wei Y, Sowers J R. Role of mitochondrial dysfunction in insulin resistance. Circ Res. 2008; 102(4):401-414.
40. Jongpiputvanich S, Sueblinvong T, Norapucsunton T. Mitochondrial respiratory chain dysfunction in various neuromuscular diseases. J Clin Neurosci. 2005; 12(4):426-428.
41. Hebert S L, Lanza I R. Nair K S. Mitochondrial DNA alterations and reduced mitochondrial function in aging. Mech Ageing Dev. 2010; 131(7-8):451-462.
42. Lanza I R, Nair K S. Mitochondrial function as a determinant of life span. Pflugers Arch. 2010; 459(2):277-289.
43. Zanoteli E, Vergani N, Campos Y, Vainzof M, Oliveira A S, d'Azzo A. Mitochondrial alterations in dynamin 2-related centronuclear myopathy. Arq Neuropsiquiatr. 2009; 67(1): 102-104.
44. Naya F J, Mercer B, Shelton J, Richardson J A, Williams R S, Olson E N. Stimulation of slow skeletal muscle fiber gene expression by calcineurin in vivo. J Biol. Chem. 2000; 275(7):4545-4548.
45. Wu H, et al. Regulation of mitochondrial biogenesis in skeletal muscle by CaMK. Science. 2002; 296(5566):349-352.
46. Millay D P, et al. Genetic and pharmacologic inhibition of mitochondrial-dependent necrosis attenuates muscular dystrophy. Nat. Med. 2008; 14(4):442-447.
47. Kim M S, et al. Protein kinase D1 stimulates MEF2 activity in skeletal muscle and enhances muscle performance. Mol Cell Biol. 2008; 28(11):3600-3609.
48. Frezza C, Cipolat S, Scorrano L. Organelle isolation: functional mitochondria from mouse liver, muscle and cultured fibroblasts. Nat. Protoc. 2007; 2(2):287-295.
49. Frisard M I, et al. Toll-like receptor 4 modulates skeletal muscle substrate metabolism. Am J Physiol Endocrinol Metab. 2010; 298(5):E988-E998.
50. Hulver M W, et al. Elevated stearoyl-CoA desaturase-1 expression in skeletal muscle contributes to abnormal fatty acid partitioning in obese humans. Cell Metab. 2005; 2(4):251-261.
51. Heilbronn L K, et al. Glucose tolerance and skeletal muscle gene expression in response to alternate day fasting. Obes Res. 2005; 13(3):574-581.
52. Oh, M., et al. Calcineurin is necessary for the maintenance but not embryonic development of slow muscle fibers. Mol Cell Biol. 2005; (25):6629-6638.

All literature, publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the present invention described herein. Such equivalents are intended to be encompassed by the appended claims.

1. A method of preventing or treating a centronuclear myopathy in a subject in need thereof comprising administering to the subject an agonist of a miR-133 family member.

2. (canceled)

3. A method of inhibiting fast-to-slow myofiber conversion in a subject in need thereof comprising administering to the subject an agonist of a miR-133 family member.

4. A method of preventing or treating a mitochondrial dysfunction in a subject in need thereof comprising administering to the subject an agonist of a miR-133 family member.

5.-6. (canceled)

7. The method of claim 1, wherein the agonist is a polynucleotide comprising a miR-133a or miR-133b sequence.

8. The method of claim 7, wherein the polynucleotide comprises a pri-miR-133a, pre-miR-133a, mature miR-133a, pri-miR-133b, pre-miR-133b, or mature miR-133b sequence.

9. The method of claim 8, wherein the polynucleotide comprises a sequence of 5′-UUUGGUCCCCUUCAACCAGCUG-3′ (SEQ ID NO: 2) or 5′-UUUGGUCCCCUUCAACCAGCUA-3′ (SEQ ID NO: 4).

10.-13. (canceled)

14. The method of claim 7, wherein said polynucleotide is encoded by an expression vector.

15. The method of claim 14, wherein said polynucleotide is under the control of a skeletal muscle promoter.

16. The method of claim 15, wherein said skeletal muscle promoter is the muscle creatine kinase promoter.

17.-22. (canceled)

23. The method of claim 1, wherein the subject has a mutation in the myotubularin (MTM1) gene, dynamin 2 (DNM2) gene, or amphiphysin 2 (BIN1) gene.

24.-31. (canceled)

32. The method of claim 3, wherein the agonist is a polynucleotide comprising a miR-133a or miR-133b sequence.

33. The method of claim 32, wherein the polynucleotide comprises a pri-miR-133a, pre-miR-133a, mature miR-133a, pri-miR-133b, pre-miR-133b, or mature miR-133b sequence.

34. The method of claim 33, wherein the polynucleotide comprises a sequence of 5′-UUUGGUCCCCUUCAACCAGCUG-3′ (SEQ ID NO: 2) or 5′-UUUGGUCCCCUUCAACCAGCUA-3′ (SEQ ID NO: 4).

35. The method of claim 32, wherein said polynucleotide is encoded by an expression vector.

36. The method of claim 35, wherein said polynucleotide is under the control of a skeletal muscle promoter.

37. The method of claim 36, wherein said skeletal muscle promoter is the muscle creatine kinase promoter.

38. The method of claim 3, wherein the subject has a mutation in the myotubularin (MTM1) gene, dynamin 2 (DNM2) gene, or amphiphysin 2 (BIN1) gene.

39. The method of claim 4, wherein the agonist is a polynucleotide comprising a miR-133a or miR-133b sequence.

40. The method of claim 38, wherein the polynucleotide comprises a pri-miR-133a, pre-miR-133a, mature miR-133a, pri-miR-133b, pre-miR-133b, or mature miR-133b sequence.

41. The method of claim 39, wherein the polynucleotide comprises a sequence of 5′-UUUGGUCCCCUUCAACCAGCUG-3′ (SEQ ID NO: 2) or 5′-UUUGGUCCCCUUCAACCAGCUA-3′ (SEQ ID NO: 4).

42. The method of claim 38, wherein said polynucleotide is encoded by an expression vector.

43. The method of claim 41, wherein said polynucleotide is under the control of a skeletal muscle promoter.

44. The method of claim 42, wherein said skeletal muscle promoter is the muscle creatine kinase promoter.

45. The method of claim 4, wherein the subject has a mutation in the myotubularin (MTM1) gene, dynamin 2 (DNM2) gene, or amphiphysin 2 (BIN1) gene.


SEQUENCE LISTING (34 sequences)

SEQ ID NO: 1 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 2 - RNA, 22 nt, Homo sapiens
uuugguccccuucaaccagcug

SEQ ID NO: 3 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 4 - RNA, 22 nt, Homo sapiens
uuugguccccuucaaccagcua

SEQ ID NO: 5 - DNA, 21 bp, Mus sp.
tccagctactgccatgctgat

SEQ ID NO: 6 - DNA, 20 bp, Mus sp.
tcgacttcctctggttccat

SEQ ID NO: 7 - DNA, 20 bp, Mus sp.
ctttgcctttgagctagacc

SEQ ID NO: 8 - DNA, 20 bp, Mus sp.
gcacgtgctctgtcttctta

SEQ ID NO: 9 - DNA, 20 bp, Mus sp.
catctgcgcatttctgtcct

SEQ ID NO: 10 - DNA, 20 bp, Mus sp.
atcatacgcttcaccgactg

SEQ ID NO: 11 - DNA, 20 bp, Mus sp.
gttatcgtcattctgctggc

SEQ ID NO: 12 - DNA, 20 bp, Mus sp.
gcctattccacagatgaagc

SEQ ID NO: 13 - DNA, 20 bp, Mus sp.
tggctcatggtcctcaagat

SEQ ID NO: 14 - DNA, 20 bp, Mus sp.
cctcagctttggctgaagat

SEQ ID NO: 15 - DNA, 20 bp, Mus sp.
agcttggagcaggtcaagaa

SEQ ID NO: 16 - DNA, 21 bp, Mus sp.
gctctacaaaggctgtaatcg

SEQ ID NO: 17 - DNA, 20 bp, Mus sp.
actcagagaaggatgcagct

SEQ ID NO: 18 - DNA, 20 bp, Mus sp.
ctctacagggtcttctagga

SEQ ID NO: 19 - DNA, 20 bp, Mus sp.
gtgtcttcagacaaggtctc

SEQ ID NO: 20 - DNA, 20 bp, Mus sp.
acccttcagaacatacaggc

SEQ ID NO: 21 - DNA, 23 bp, Mus sp.
ccttggcaccaatgtcccggctc

SEQ ID NO: 22 - DNA, 22 bp, Mus sp.
gaagcgcaatgcagagtcggtg

SEQ ID NO: 23 - DNA, 18 bp, Mus sp.
atgagctccgacgccgag

SEQ ID NO: 24 - DNA, 24 bp, Mus sp.
tctgttagcatgaactggtaggcg

SEQ ID NO: 25 - DNA, 23 bp, Mus sp.
aaggagcaggacaccagcgccca

SEQ ID NO: 26 - DNA, 23 bp, Mus sp.
atctctttggtcactttcctgct

SEQ ID NO: 27 - DNA, 22 bp, Mus sp.
gtgatttctcctgtcacctctc

SEQ ID NO: 28 - DNA, 23 bp, Mus sp.
ggaggaccgcaagaacgtgctga

SEQ ID NO: 29 - RNA, 21 nt, Unknown
  miR-133a sequence
uugguccccuucaaccagcua

SEQ ID NO: 30 - RNA, 29 nt, Mus sp.
ugcccuccaugcugggaccaggcuccccg

SEQ ID NO: 31 - RNA, 28 nt, Homo sapiens
cgccccuaugcugggaccaggcucccag

SEQ ID NO: 32 - RNA, 29 nt, Rattus sp.
ugccccccaugcugggaccaggcuccccg

SEQ ID NO: 33 - RNA, 7 nt, Unknown
  Conserved miR-133a binding site
gggacca

SEQ ID NO: 34 - RNA, 7 nt, Unknown
  miR-133a seed sequence
ugguccc